(19) United States
(12) Patent Application Publication
(10) Pub. No.: US 2025/0276015 A1
(43) Pub. Date: Sep. 4, 2025

(54) RECOMBINANT POLYPEPTIDES COMPRISING SINGLE-DOMAIN ANTIBODIES TARGETING HERV-K SUBTYPE HML-2

(71) Applicant: The United States of America, as represented by the Secretary, Dept. of Health and Human Services, Bethesda, MD (US)

(72) Inventors: Avindra Nath, Silver Spring, MD (US); Wenxue Li, Potomac, MD (US); Kevon Sampson, Kensington, MD (US); Ashish Shah, Coral Gables, FL (US); Naomi Taylor, Washington, DC (US); Saliha Majdoul, Rockville, MD (US)

(21) Appl. No.: 18/859,027

(22) PCT Filed: Apr. 26, 2023
(86) PCT No.: PCT/US2023/066224

§ 371 Date: Oct. 22, 2024

Related U.S. Application Data
(60) Provisional application No. 63/335,824, filed on Apr. 28, 2022.

(51) Int. Cl. A61K 35/17; A61K 40/11; A61K 40/31; A61K 40/46; C07K 14/705; C07K 14/725; C07K 16/10; C12N 5/0783; C12N 15/86

(52) U.S. Cl. CPC A61K 35/17; A61K 40/11; A61K 40/31; A61K 40/46; C07K 14/7051; C07K 14/70517; C07K 14/70521; C07K 14/70578; C07K 16/1036; C12N 5/0636; C12N 15/86; A61K 2239/13; A61K 2239/17; A61K 2239/21; C07K 2317/22; C07K 2317/24; C07K 2317/53; C07K 2317/565; C07K 2317/567; C07K 2317/569; C07K 2317/92; C07K 2319/02; C07K 2319/03; C07K 2319/21; C07K 2319/41; C07K 2319/43; C07K 2319/60; C12N 2510/00; C12N 2740/15043

(57) ABSTRACT

The disclosure provides recombinant polypeptides that specifically bind to an envelope epitope of HERV-K HML-2, wherein such engineered polypeptides may be single-domain antibodies or immunoglobulin variable domains. The disclosure also provides CAR comprising such recombinant polypeptides. The disclosure further provides nucleic acid molecules that encode such recombinant polypeptides or CARs, and methods of making such recombinant polypeptides or CARs. The disclosure further provides pharmaceutical compositions that comprise such recombinant polypeptides or CARs, and methods of treatment using such recombinant polypeptides or CARs.

### CROSS REFERENCE TO RELATED APPLICATIONS

This application claims the benefit of U.S. Provisional Application No. 63/335,824, filed Apr. 28, 2022, which is incorporated by reference herein in its entirety.

### SEQUENCE LISTING

A computer readable form of the Sequence Listing is filed with this application by electronic submission and is incorporated into this application by reference in its entirety. The Sequence Listing is contained in the XML file created on Apr. 19, 2023, having the file name “22-0641-WO_Sequence-Listing.xml” and is 29 kilobytes in size.

### GOVERNMENT RIGHTS

This invention was made with Government support. The Government has certain rights in the invention.

### BACKGROUND OF THE INVENTION

### Field of the Invention

This disclosure generally relates to recombinant polypeptides that specifically bind to a conformational epitope on the envelope of HERV-K subtype HML-2, wherein such recombinant polypeptides may be single-domain antibodies or nanobodies. The disclosure also relates to compositions and/or pharmaceutical compositions that comprise such recombinant polypeptides, and diagnostic methods and/or methods of treatment using such recombinant polypeptides.

### Description of Related Art

Monoclonal antibodies (mAbs) are widely used as therapeutics for clinical treatment. There are over 100 FDA-approved mAbs products (1). However, their large size hinders their efficacy and cost-effectiveness (2). Antibodies found in camelids consists of only two heavy chains. Each heavy chain has a single variable domain (VHH). Their antigen specificity is determined by three complementarity determining regions (CDRs) at the exposed end of VHH. When the VHH is isolated, it still preserves the full antigen-binding property. Because of its small size of about 15 kDa, it was coined the name “nanobody” (3). Its intrinsic nanoscale gives it advantage for deep tissue penetration, as well as potentially blood brain barrier crossing (4). One potential use of nanobodies is in cancer diagnosis and therapy (5). For example, chimeric antigen receptor (CAR) T cell therapeutics based on nanobodies can offer better solid tumor penetration and higher efficacy for anti-tumor response.

The human genome has up to 8% sequences of retroviral origin. These retroviral elements are largely defective and inactive because of mutations and deletions accumulated during human evolution. However, some of the most recently acquired retroviral sequences, named human endogenous retrovirus-K (HERV-K), have preserved complete viral genome structure and intact open reading frames. They may express certain viral proteins or form virus like particles under certain physiological and pathological conditions. HERV-K activation has been associated with a large number of diseases including cancer, immune disorders, and neurodegenerative diseases (6).

The potential implication of HERV-K in the neuropathology of amyotrophic lateral sclerosis (ALS) has been studied for decades. ALS, also called “Lou Gehrig's disease”, is a progressive neurodegenerative disorder primarily affecting motor cortex and spinal cord. It is a debilitating disease with half of patients dying in two to five years. HERV-K viral genes are activated in the cortex and spinal cord in a subgroup of ALS patients (7). Further study found that transfection of human neuron culture with either whole HERV-K genomic sequence or HERV-K envelope protein (Env) caused significant cell toxicity. A transgenic mouse model expressing HERV-K Env in neurons showed specific motor neuron toxicity and subsequent motor function disfunction.

Furthermore, HERV-K activation and Env expression were also associated with many different types of tumor like teratocarcinoma, germ cell tumors, melanoma, ovarian, breast, brain and prostate cancer (8-11), as well as cancer cell lines (12-16). HERV-K Env may be specifically associated with tumorigenesis (16). Knocking down HERV-K using env-targeting shRNA can significantly reduce growth rate and cause cell death in cancer cells with high HERV-K activity (15, 17).

There are 8 HERV-K loci in the human genome that have intact open reading frame to express full-length Env protein. Because Env plays a critical role not only in the packaging and infection of viral particles, but in the neurotoxicity and tumorigenesis, it is a potential target for therapeutics development.

As disclosed herein, a HERV-K Env specific nanobody, called G10, was developed and showed high affinity binding of Env protein in various experimental applications. The antibodies as disclosed herein fill a need for high efficacy therapeutics against wide range of diseases from cancer to neurodegenerative disorders involving HERV-K activation and Env expression. The antibodies as disclosed herein fill a need for diagnostic tools either in vivo as a ligand for identifying tumor cells or ex-vivo for identification of tumor antigen in biological materials.

### SUMMARY OF THE INVENTION

It is against the above background that the present invention provides certain advantages over the prior art.

Although this invention as disclosed herein is not limited to specific advantages or functionalities (such for example, the ability to make recombinant polypeptide that specifically binds to an envelope epitope of HERV-K subtype HML-2 that can be used in pharmaceutical composition in methods of treating at least one condition mediated by HERV-K subtype HML-2), the invention provides recombinant polypeptide that specifically binds to an envelope epitope of HERV-K subtype HML-2, wherein the polypeptide comprises:
(a) the amino acid sequence of SEQ ID NOs: 3 or 4, or a fragment thereof;
(b) an amino acid sequence that is at least 85% identical to the amino acid sequence of SEQ ID NOs: 3 or 4; or
(c) complementarity determining regions, CDR1, CDR2, and CDR3, wherein:
   (i) CDR1 comprises the amino acid sequence of SEQ ID NO:5, or an amino acid sequence that is at least 85% identical to the amino acid sequence of SEQ ID NO:5;
   (ii) CDR2 comprises the amino acid sequence of SEQ ID NO:6, or an amino acid sequence that is at least 85% identical to the amino acid sequence of SEQ ID NO:6; and
   (iii) CDR3 comprises the amino acid sequence of SEQ ID NO:7, or an amino acid sequence that is at least 85% identical to the amino acid sequence of SEQ ID NO:7.

In one aspect of the recombinant polypeptide disclosed, the recombinant polypeptide comprises the amino acid sequence of SEQ ID NO:3.

In one aspect of the recombinant polypeptide disclosed, the recombinant polypeptide is a single-domain antibody.

In one aspect of the recombinant polypeptide disclosed, the single-domain antibody is a humanized single domain antibody.

In one aspect of the recombinant polypeptide disclosed, the recombinant polypeptide comprises a VHH sequence.

In one aspect of the recombinant polypeptide disclosed, the VHH sequence comprises camelized or llamaized framework regions of a human VH.

In one aspect of the recombinant polypeptide disclosed, the recombinant polypeptide further comprises at least one therapeutic agent or imaging moiety.

In one aspect of the recombinant polypeptide disclosed, the recombinant polypeptide is either directly linked to the at least one therapeutic agent or imaging moiety, or is linked to the at least one therapeutic agent or imaging moiety via a linker or spacer.

In one aspect of the recombinant polypeptide disclosed, the linker comprises an amino acid sequence.

In one aspect of the recombinant polypeptide disclosed, the linker amino acid sequence comprises between about 1-25 amino acids, about 1-20 amino acids, about 1-15 amino acids, about 1-10 amino acids, about 1-5 amino acids, about 1-4 amino acids, about 1-3 amino acids, about 2 amino acids, or one (1) amino acid.

In one aspect of the recombinant polypeptide disclosed, the linker comprises an amino acid sequence selected from the group consisting of (GGS)n, (GGGS)n (SEQ ID NO:9), and (GGGGS)n (SEQ ID NO:10), wherein n=1-5.

In one aspect of the recombinant polypeptide disclosed, the at least one therapeutic agent comprises a therapeutic agent selected from the group consisting of chemotherapeutic agents, immunotherapeutic agents, radioactive agents, or a biologic agents.

In one aspect of the recombinant polypeptide disclosed, the at least one imaging moiety is selected from the group consisting of radiolabels, fluorescent labels, enzymatic labels, or PET imaging agents.

The invention also provides a pharmaceutical composition comprising a therapeutically effective amount of the recombinant polypeptide disclosed herein and a pharmaceutically acceptable carrier.

The invention also provides a method of treating at least one condition mediated by HERV-K subtype HML-2, comprising administering the pharmaceutical composition disclosed herein to a patient in need of such treatment.

In one aspect of the methods disclosed, the at least one condition mediated by HERV-K subtype HML-2 is selected from cancer (breast, brain, prostate, melanoma, germ cell tumors, ovarian, pancreatic, testes, glioblastoma, teratocarcinoma, medulloblastoma, lung, hepatocellular, colorectal, sarcoma, lymphoma, and/or metastases thereof), neurodegenerative diseases (ALS, Jacob Creutzfeldt Disease, Alzheimer's disease, and Frontotemporal dementia), or immune diseases (Rheumatoid arthritis, myalgic encephalomyelitis/chronic fatigue syndrome, and Lupus) that express HERV-K subtype HML-2.

The invention also provides an isolated nucleic acid molecule comprising a nucleotide sequence encoding the recombinant polypeptide disclosed herein.

The invention also provides an expression vector comprising the nucleic acid molecule disclosed herein.

The invention also provides an isolated host cell comprising the nucleic acid molecule disclosed herein.

The invention also provides isolated host cell comprising the expression vector disclosed herein.

In one aspect of the isolated host cell disclosed herein, the host cell is a mammalian cell or an insect cell.

The invention also provides a method for making the recombinant polypeptide that specifically binds to one or more envelope epitopes of HERV-K subtype HML-2, comprising expressing in a host cell the isolated nucleic acid molecule disclosed herein.

The invention also provides a method for making a recombinant polypeptide that specifically binds to one or more envelope epitopes of HERV-K subtype HML-2, comprising expression in a host cell at least one nucleic acid molecule comprising a nucleotide sequence encoding a polypeptide that comprises:
(a) the amino acid sequence of SEQ ID NOs: 3 or 4, or a fragment thereof;
(b) an amino acid sequence that is at least 85% identical to the amino acid sequence of SEQ ID NOs: 3 or 4; or
(c) complementarity determining regions, CDR1, CDR2, and CDR3, wherein:
   (i) CDR1 comprises the amino acid sequence of SEQ ID NO:5, or an amino acid sequence that is at least 85% identical to the amino acid sequence of SEQ ID NO:5;
   (ii) CDR2 comprises the amino acid sequence of SEQ ID NO:6, or an amino acid sequence that is at least 85% identical to the amino acid sequence of SEQ ID NO:6; and
   (iii) CDR3 comprises the amino acid sequence of SEQ ID NO:7, or an amino acid sequence that is at least 85% identical to the amino acid sequence of SEQ ID NO:7.

The invention also provides an in vitro method of detecting the presence of one or more envelope epitopes of HERV-K subtype HML-2, comprising the steps of:
(a) obtaining a sample from a subject;
(b) incubating the sample with the recombinant polypeptide disclosed herein;
(c) detecting the binding of the recombinant polypeptide in the sample; and
(d) comparing the binding detected in step (c) with a standard,
wherein a difference in binding relative to the standard indicates the presence of one or more envelope epitopes of HERV-K subtype HML-2 in the sample.

The invention also provides a method of detecting the presence of one or more envelope epitopes of HERV-K subtype HML-2 in a patient, comprising the steps of:
(a) administering to the patient the recombinant polypeptide disclosed herein;
(b) detecting the binding of the recombinant polypeptide in the patient; and
(c) comparing the binding detected in step (b) with a standard;
wherein a difference in binding relative to the standard indicates the presence of one or more envelope epitopes of HERV-K subtype HML-2 in the patient.

The invention also provides a chimeric antigen receptor (CAR), wherein the CAR comprises the recombinant polypeptide disclosed herein.

The invention also provides a chimeric antigen receptor (CAR), wherein the CAR comprises:
(a) an extracellular binding domain that specifically binds to one or more envelope epitopes of HERV-K subtype HML-2;
(b) a transmembrane domain; and
(c) at least one cytoplasmic signaling domain.

In one aspect of the CAR disclosed herein, the extracellular binding domain comprises a single-domain antibody that specifically binds to one or more envelope epitopes of HERV-K subtype HML-2.

In one aspect of the CAR disclosed herein, the single-domain antibody comprises an amino acid sequence sharing at least 85% sequence identity to the amino acid sequence of SEQ ID NO:3.

In one aspect of the CAR disclosed herein, the single-domain antibody comprises complementarity determining regions, CDR1, CDR2, and CDR3, wherein:
(a) CDR1 comprises the amino acid sequence of SEQ ID NO:5, or an amino acid sequence that is at least 85% identical to the amino acid sequence of SEQ ID NO: 5;
(b) CDR2 comprises the amino acid sequence of SEQ ID NO:6, or an amino acid sequence that is at least 85% identical to the amino acid sequence of SEQ ID NO: 6; and
(c) CDR3 comprises the amino acid sequence of SEQ ID NO:7, or an amino acid sequence that is at least 85% identical to the amino acid sequence of SEQ ID NO: 7.

In one aspect of the CAR disclosed herein, the transmembrane domain comprises an amino acid sequence derived from a molecule that is the alpha chain of the T-cell receptor, the beta chain of the T-cell receptor, the zeta chain of the T-cell receptor, CD3-epsilon, CD3-zeta, CD4, CD5, CD8, CD9, CD16, CD22, CD28, CD33, CD37, CD45, CD64, CD80, CD86, CD134, CD137, or CD154.

In some embodiments, the transmembrane domain comprises an amino acid sequence derived from CD8 or CD28.

In one aspect of the CAR disclosed herein, the at least one cytoplasmic signaling domain comprises at least one amino acid sequence derived from a molecule that is CD2, CD3-zeta, CD3-gamma, CD3-delta, CD3-epsilon, CD5, CD7, CD22, CD27, CD28, CD30, CD40, CD66d, CD79a, CD79b, 4-1BB (CD137), OX40, PD-1, ICOS, lymphocyte function-associated antigen-1 (LFA-1), LIGHT, NKG2C, B7-H3, FcR-gamma, FcR-beta, or TCR-zeta.

In some embodiments, the at least one cytoplasmic signaling domain comprises at least one amino acid sequence derived from CD3-zeta, CD28, or 4-1BB.

In one aspect of the CAR disclosed herein, the CAR further comprises a hinge domain selected from CD8 or CD28.

The invention also provides a population of T-cells comprising the chimeric antigen receptor (CAR) disclosed herein.

In one aspect of the population disclosed herein, the population of T-cells is present in a therapeutically effective amount for treatment of at least one condition mediated by HERV-K subtype HML-2.

The invention also provides a pharmaceutical composition comprising a population of T-cells expressing the CAR according disclosed herein, and a pharmaceutically acceptable carrier.

In one aspect of the pharmaceutical composition disclosed herein, the composition further comprises at least one therapeutic agent.

In one aspect of the pharmaceutical composition disclosed herein, the at least one therapeutic agent comprises a therapeutic agent selected from the group consisting of cytotoxic agents, chemotherapeutic agents, immunotherapeutic agents, radioactive agents, or a biologic agents.

The invention also provides an isolated nucleic acid comprising a nucleotide sequence encoding the CAR disclosed herein.

The invention also provides a method of inducing a T-cell response in a subject suffering from at least one condition mediated by HERV-K subtype HML-2, wherein the method comprises administering to the subject a therapeutically effective amount of the population of T-cells disclosed herein, or the pharmaceutical composition disclosed herein, wherein the administration induces an immune response to the at least one condition mediated by HERV-K subtype HML-2.

In one aspect of the methods disclosed herein, the at least one condition mediated by HERV-K subtype HML-2 is selected from cancer (breast, brain, prostate, melanoma, germ cell tumors, ovarian, pancreatic, testes, glioblastoma, teratocarcinoma, medulloblastoma, lung, hepatocellular, colorectal, sarcoma, lymphoma, and/or metastases thereof), neurodegenerative diseases (ALS, Jacob Creutzfeldt Disease, Alzheimer's disease, and Frontotemporal dementia), or immune diseases (Rheumatoid arthritis, myalgic encephalomyelitis/chronic fatigue syndrome, and Lupus) that express HERV-K subtype HML-2.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1: Diagram to show the construct that expresses the HERV-K Env nanobody G10. The construct includes a 6×His tag for recombinant protein purification with immobilized metal affinity chromatography, and a 3×Myc tag for nanobody detection with an anti-Myc antibody.

FIG. 2: Diagram to show the construct that expresses a modified version of G10 (G10-Tat). In this modified construct, a cell penetrating peptide Tat (amino acid sequence RKKRRQRRR; SEQ ID NO:8) was incorporated, while the tags were re-arranged.

FIG. 3: Immunoprecipitation with nanobodies. The transfected HERV-K Env protein from HEK293T cells were immunoprecipitated with different nanobodies (A03, B01, G10, A11, F02, and G02) and anti-Myc magnetic beads. The precipitates were detected with anti-Env monoclonal antibody by Western blot. In both panels, the pcDNA(−) lane is a WB negative control. The Env(+) lane is a WB positive control. The Env+beads lane is from precipitation with nanobody omitted. Following paired lanes compare precipitation result from negative control (pcDNA) to Env transfected cells. The full length (about 85 kDa) and transmembrane subunit (about 45 kDa) Env bands are marked.

FIG. 4: Immunofluorescent staining of Env-GFP with G10. HEK293T cells were transfected with Env-GFP plasmid and stained with G10. The Env-GFP expression can be observed in GFP channel without staining, while it can also be observed in Texas Red channel after staining with G10.

FIG. 5: Flow cytometry plots of HEK293 cells transfected with Env-GFP. The top panels showed the negative result of cells without Env-GFP. As a negative staining control, G10 staining is omitted (anti-Myc mAb). As a positive staining control, a commercial anti-HERV-K Env mAb was used for staining for flow cytometry. Cells were gated with GFP channel for Env-GFP detection (x-axis) and Alexa Fluor 647 channel for antibody binding of Env (y-axis).

FIG. 6: Antigen binding region (ABR) identified with Paratome. Using Alphafold2, the 3D structure of G10 was modeled with the ABR region highlighted.

FIG. 7: Measurement of the affinity of G10 nanobody binding to HERV-K Env protein. Recombinant G10 and HERV-K Env proteins were used for the protein thermal shift assay. Interaction of HERV-K Env protein with G10 using protein thermal shift assays. The relative fluorescence emitted by the thermal shift dye is recorded during the temperature ramp phase and plotted versus temperature (left panel). Tm value was calculated from the derivative of the thermal melt curves and plotted as effects of various concentrations of G10. The calculated ED₅₀ is 0.243 nm.

FIG. 8: Identification of a HERV-K Env epitope (SEQ ID NO: 11) for G10 binding. Epitope fingerprinting technique by EPITOPIC GmbH revealed that an epitope can comprise 310-FYPWEW (SEQ ID NO:12) and 361-ETRDRKPFYT (SEQ ID NO:13) as boxed in FIG. 8, which are in the surface unit (SU) domain of HERV-K Env.

FIG. 9: Alphafold simulation showing that the epitope (in “ball and stick” model) forms two loops in very close proximity.

FIG. 10: Generation of G10 VHH CAR-T lentiviral constructs. Four distinct G10 VHH nanobody-derived CAR constructs were produced and schema of the different constructs are presented. Constructs contained either the CD8 hinge/transmembrane domain and the 4-1BB co-stimulatory domain (top) or the CD28 hinge/transmembrane domain and the CD28 co-stimulatory domain (bottom). For each construct type, a second construct harboring a 3×Flag sequence at the N-terminus were generated. To facilitate evaluation of T cell transduction, all constructs were generated to express eGFP downstream of the T2A ribosomal skipping sequence.

FIG. 11: Transduction of primary T cells with the generated G10 VHH CAR constructs. Human T cells were transduced with the lentiviral vectors described in FIG. 10 using VSV-G-pseudotyped virus at a multiplicity of infection (MOI) of 5. Transduction of all constructs was monitored as a function of GFP expression and for CAR constructs harboring the 3×Flag tag, cell surface CAR expression was evaluated by anti-Flag antibody staining. Representative flow cytometry plots are presented.

FIG. 12: Functional evaluation of G10 VHH CAR T-cells as a measure of cytokine secretion. G10 VHH CAR T cell function was evaluated by co-culture with HERV-KEnv-positive HEK 293T cells (light grey) or control non-transfected (NT) HEK293T (dark grey). Co-cultures were performed at an effector/target (E/T) ratio of 1:1 with CAR T-cell numbers evaluated as a function of GFP expression. Mock-transduced T cells were used as a negative control. Production of IFN-gamma and IL-2 production were measured using a Cytokine Bead Array at 24 hours and mean cytokine levels±SEM for each condition are presented for two independent donors.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE DISCLOSURE

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

Before describing the present invention in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The terms “comprises” and variations thereof do not have a limiting meaning where these terms appear in the description and claims.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Ranges and amounts can be expressed as “about” a particular value or range. About can also include the exact amount. Typically, the term “about” includes an amount that would be expected to be within experimental error. The term “about” includes values that are within 10% less to 10% greater of the value provided.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA). Standard recombinant DNA methodologies are used to construct the polynucleotides of the disclosure, incorporate such polynucleotides into recombinant expression vectors, and introduce such vectors into host cells to produce the amino acid sequences, proteins, and polypeptides of the disclosure. See e.g., Sambrook et al., 2001, MOLECULAR CLONING: A LABORATORY MANUAL (Cold Spring Harbor Laboratory Press, 3rd ed.). Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

As used herein, the terms “polynucleotide,” “nucleotide,” “oligonucleotide,” and “nucleic acid” can be used interchangeably to refer to nucleic acid comprising deoxyribonucleic acid (DNA), ribonucleic acid (RNA), derivatives thereof, or combinations thereof, in either single-stranded or double-stranded embodiments depending on context as understood by the skilled worker. DNA can have one or more bases selected from the group consisting of adenine (symbol “A”), thymine (symbol “T”), cytosine (symbol “C”), or guanine (symbol “G”), and a ribonucleic acid can have one or more bases selected from the group consisting of adenine (symbol “A”), uracil (symbol “U”), cytosine (symbol “C”), or guanine (symbol “G”). Nucleic acids can also have the following IUPAC symbols:

TABLE 1
Nucleic acid IUPAC symbols.

| | | Bases | | | | Complementary |
|---|---|---|---|---|---|---|
| Description | Symbol | represented | | | | base |
| Weak | W | A | | | T | W |
| Strong | S | | C | G | | S |
| Amino | M | A | C | | | K |
| Keto | K | | | G | T | M |
| Purine | R | A | | G | | Y |
| Pyrimidine | Y | | C | | T | R |
| Not A | B | | C | G | T | V |
| Not C | D | A | | G | T | H |
| Not G | H | A | C | | T | D |
| Not T | V | A | C | G | | B |
| Any one base | N | A | C | G | T | N |

As used herein, antibody or immunoglobulin also includes “single-domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered or recombinant single-domain antibodies. The variable heavy chain of single-domain antibodies devoid of light chains are known in the art as “VHH” or “nanobody” and as used herein a single-domain antibody can refer to a nanobody. Single domain antibodies may be derived from any species including, but not limited to mouse, human, camel, llama, goat, rabbit, and bovine. Single domain antibodies may be naturally occurring single domain antibodies known as heavy chain antibody devoid of light chains. In particular, Camelidae species, for example camel, dromedary, llama, alpaca and guanaco, produce heavy chain antibodies naturally devoid of light chain.

The variable heavy chain of single-domain antibodies devoid of light chains are known in the art as “VHH” or “nanobody”. Similar to conventional VH domains, VHHs contain four FRs and three CDRs. Nanobodies have advantages over conventional antibodies: they are smaller than IgG molecules, and as a consequence properly folded functional nanobodies can be produced by in vitro expression while achieving high yield. For example, VHH domains, Nanobodies and proteins/polypeptides containing the same can be produced using microbial fermentation and do not require the use of mammalian expression systems; VHH domains and nanobodies are relatively small (approximately 15 kDa, or 10 times smaller than a conventional IgG), and therefore show high (er) penetration into tissues (including but not limited to solid tumors and other dense tissues) than such conventional 4-chain antibodies and antigen-binding fragments thereof; VHH domains and nanobodies can show so-called cavity-binding properties (inter alia due to their extended CDR3 loop, compared to conventional VH domains) and can therefore also access targets and epitopes not accessible to conventional 4-chain antibodies and antigen-binding fragments thereof. Furthermore, nanobodies are very stable, and resistant to the action of proteases.

As used herein, “VHH domain” refers to variable domains present in naturally occurring heavy-chain antibodies, in order to distinguish them from the heavy chain variable domains that are present in conventional four-chain antibodies (referred to herein as “VH domains”) and from the light chain variable domains that present in conventional four-chain antibodies (referred to herein as “VL domains”).

VHH domains have a number of unique structural characteristics and functional properties which make isolated VHH domains (as well as single-domain antibodies, which are based on VHH domains and which share these structural characteristics and functional properties with the naturally occurring VHH domains) and proteins containing the VHH domains highly advantageous for use as functional antigen-binding domains or proteins. For example, VHH domains (which have been optimized by nature to functionally bind to an antigen without the presence of, or interaction with, a light chain variable domain) and single-domain antibodies can function as a single, relatively small, functional antigen-binding structural unit, domain, or protein. This distinguishes VHH domains from the VH and VL domains of conventional four-chain antibodies, which by themselves are generally not suited for practical application as single antigen-binding proteins or domains, but need to be combined in some form or another to provide a functional antigen-binding unit (as in for example conventional antibody fragments such as Fab fragments and ScFv fragments, the latter of which consist of a VH domain covalently linked to a VL domain).

The term “single-domain antibody,” as used herein in its broadest sense, is not limited to a specific biological source or to a specific method of preparation. A single-domain antibody can be obtained by (1) isolating the VHH domain of a naturally occurring heavy chain antibody; (2) expressing a nucleotide sequence encoding a naturally occurring VHH domain; (3) “humanization” of a naturally occurring VHH domain or by expression of a nucleic acid encoding such humanized VHH domain; (4) “camelization” of a naturally occurring VH domain from any animal species, in particular a species of mammal, such as from a human being, or by expression of a nucleic acid encoding such a camelized VH domain; (5) “camelisation” of a “domain antibody” or “Dab” as described by Ward et al., 1989, Nature 341:544, or by expression of a nucleic acid encoding such a camelized VH domain; (6) using synthetic or semi-synthetic techniques for preparing amino acid sequences, proteins, or polypeptides; (7) preparing a nucleic acid encoding a single-domain antibody using techniques for nucleic acid synthesis, followed by expression of the nucleic acid thus obtained; and/or (8) any combination of the above.

In some embodiments, the recombinant polypeptides of the disclosure correspond to amino acid sequences of naturally occurring VHH domains, but that have been “humanized,” i.e., by replacing one or more amino acid residues in the amino acid sequence of the naturally occurring VHH sequence by one or more of the amino acid residues that occur at the corresponding positions in a VH domain from a conventional four-chain antibody from a human being. This can be performed in a manner known in the art.

In some embodiments, the recombinant polypeptides of the disclosure correspond to amino acid sequences of naturally occurring VH domains that have been “camelized,” i.e., by replacing one or more amino acid residues in the amino acid sequence of a naturally occurring VH domain from a conventional four-chain antibody by one or more of the amino acid residues that occur at the corresponding positions in a VHH domain of a heavy chain antibody. This can be performed in a manner known in the art, for example, as described in International Publication No. WO 94/04678. Such camelization may preferentially occur at amino acid positions which are present at the VH-VL interface and at the so-called Camelidae hallmark residues (see e.g., International Publication No. WO 94/04678). Preferably, the VH domain or sequence that is used as a starting material or starting point for generating or designing the camelized sequence is preferably a VH sequence from a mammal, more preferably the VH sequence of a human being. However, it should be noted that such camelized sequences can be obtained in any suitable manner known in the art and thus are not strictly limited to polypeptides that have been obtained using a polypeptide that comprises a naturally occurring VH domain as a starting material.

According to some embodiments, the recombinant polypeptides herein specifically binds to an envelope epitope of HERV-K subtype HML-2.

In one embodiment, the disclosure provides recombinant polypeptide sequences, such as immunoglobulin sequences (in some embodiments, VHH antibody sequences) that are capable of binding to an envelope epitope of HERV-K subtype HML-2, wherein the immunoglobulin sequence comprises four framework regions (FR1, FR2, FR3, and FR4) and three complementarity determining regions (CDR1, CDR2, and CDR3), wherein:
a) CDR1 is the amino acid sequence of SEQ ID NO:5; or selected from the group consisting of amino acid sequences that have at least 85%, or at least 90%, or at least 95%, or at least 99% sequence identity with the amino acid sequence of SEQ ID NO:5; or from the group consisting of amino acid sequences that have 2 or only 1 amino acid differences as compared to the amino acid sequence of SEQ ID No:5;
b) CDR2 is the amino acid sequence of SEQ ID NO:6; or selected from the group consisting of amino acid sequences that have at least 85%, or at least 90%, or at least 95%, or at least 99% sequence identity with the amino acid sequence of SEQ ID NO:6; or from the group consisting of amino acid sequences that have 2 or only 1 amino acid differences as compared to the amino acid sequence of SEQ ID No:6;
c) CDR3 is the amino acid sequence of SEQ ID NO:7; or selected from the group consisting of amino acid sequences that have at least 85%, or at least 90%, or at least 95%, or at least 99% sequence identity with the amino acid sequence of SEQ ID NO:7; or from the group consisting of amino acid sequences that have 2 or only 1 amino acid differences as compared to the amino acid sequence of SEQ ID No:7;
and in which the framework sequences may be any suitable framework sequences, such as the framework sequences of a single-domain antibody and in particular of a VHH antibody.

In another embodiment, the disclosure provides a VHH antibody amino acid sequence having at least 85%, or at least 90%, or at least 95%, or at least 99% sequence identity with at least one of the amino acid sequences of SEQ ID NOs: 3 or 4.

In another embodiment, an amino acid sequence, protein, or recombinant polypeptide disclosed herein is a VHH antibody, which has at least 80%, or at least 90%, or at least 95%, or at least 99% sequence identity with at least one of the amino acid sequences of SEQ ID NOs: 3 or 4.

An “antibody” also called “immunoglobulin” may be a natural or conventional antibody in which two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

The term “monoclonal antibody” or “mAb” as used herein refers to an antibody molecule of a single amino acid composition that is directed against a specific antigen, and is not to be construed as requiring production of the antibody by any particular method. A monoclonal antibody may be produced by a single clone of B cells or hybridoma, but may also be recombinant, i.e., produced by protein engineering.

The term “chimeric antibody” refers to a recombinant antibody or to an engineered antibody which in its broadest sense contains one or more regions from one antibody and one or more regions from one or more other antibody(ies). In particular a chimeric antibody comprises a VH domain and a VL domain of an antibody derived from a non-human animal, in association with a CH domain and a CL domain of another antibody, in particular a human antibody. As the non-human animal, any animal such as camel, llama, mouse, rat, hamster, rabbit or the like can be used. A chimeric antibody may also denote a multispecific antibody having specificity for at least two different antigens.

The term “humanized antibody” refers to an antibody which is wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains.

Numerous methods for humanization of an antibody sequence are known in the art; see e.g., the review by Almagro & Fransson (2008) Front Biosci. 13:1619-1633. One commonly used method is CDR grafting, or antibody reshaping, which involves grafting of the CDR sequences of a donor antibody, generally a mouse antibody, into the framework scaffold of a human antibody of different specificity. Because CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from for example a murine or rat antibody a fully human antibody conserving the epitope and binding characteristics of the parental antibody. A further method of humanization is the so-called 4D humanization. The 4D humanization protocol is described in WO 2009/032661A1, which is incorporated by reference herein in its entirety.

By “purified” and “isolated” it is meant, when referring to a polypeptide (i.e., the recombinant polypeptide as disclosed herein) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present. An “isolated” nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition. The terms “substantially pure” or “substantially purified,” as used herein, refer to a compound or species that is the predominant species present (i.e., on a molar basis it is more abundant than any other individual species in the composition). In some embodiments, a substantially purified fraction is a composition wherein the species comprises at least about 50% (on a molar basis) of all macromolecular species present. In other embodiments, a substantially pure composition will comprise more than about 80%, 85%, 90%, 95%, or 99% of all macromolar species present in the composition. In still other embodiments, the species is purified to essential homogeneity (contaminant species cannot be detected in the composition by conventional detection methods) wherein the composition consists essentially of a single macromolecular species.

The terms “antigen” or “antigen target,” as used herein, refers to a molecule or a portion of a molecule that is capable of being bound to the amino acid sequences, proteins, or polypeptides disclosed herein, and additionally is capable of being used in an animal to produce antibodies capable of binding to an epitope of that antigen. An antigen may have one or more epitopes.

An “epitope” is a region of an antigen that is bound by an antibody, immunoglobulin, or immunologically functional immunoglobulin fragment, such as an amino acid sequence, protein, or recombinant polypeptide of the disclosure. An antibody, immunoglobulin, or immunologically functional immunoglobulin fragment, such as the amino acid sequences, proteins, or polypeptides disclosed herein, is said to specifically bind an antigen when it preferentially recognizes its antigen target in a complex mixture of proteins and/or macromolecules. The term ‘specifically binds,’ as used herein, refers to the ability of an amino acid sequence, protein, or recombinant polypeptide of the disclosure to bind to an antigen containing an epitope with an K_{d} of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to one or more epitopes with an affinity that is at least two-fold greater than its affinity for a nonspecific antigen.

The terms “activity,” “biological activity,” or “biological property,” as used in reference to the amino acid sequences, proteins, and recombinant polypeptides of the disclosure, include, but are not limited to, epitope affinity and specificity, ability to antagonize the activity of an antigen target, the in vivo stability of the amino acid sequences, proteins, and recombinant polypeptides of the disclosure, and the immunogenic properties of the amino acid sequences proteins, and recombinant polypeptides of the disclosure. Other identifiable biological properties include, for example, cross-reactivity, (i.e., with non-human homologs of the antigen target, or with other antigen targets or tissues, generally), and ability to preserve high expression levels of protein in mammalian cells. The aforementioned properties or characteristics can be observed or measured using art-recognized techniques.

The term “K_{d},” as used herein, refers to the dissociation constant of the interaction between an amino acid sequence, protein, or recombinant polypeptide disclosed herein and an antigen target. When an amino acid sequence, protein, or recombinant polypeptide of the disclosure is a monovalent immunoglobulin sequence (for example, a monovalent VHH antibody), the monovalent immunoglobulin sequence preferably binds to serum albumin with a dissociation constant (K_{d}) of 10⁻⁵ to 10⁻¹² moles/liter or less, or 10⁻⁷ to 10⁻¹² moles/liter or less, or 10⁻³ to 10⁻¹² moles/liter, and/or with a binding affinity of at least 10⁷ M-1, or at least 10⁸ M-1, or at least 10⁹ M-1, or at least 10¹² M-1. Any K_{d} value greater than 10⁻⁴ liters/mole is generally considered to indicate non-specific binding. In some embodiments, a monovalent immunoglobulin sequence of the disclosure will bind to a desired antigen with an affinity less than 500 mM, or less than 200 nM, or less than 10 nM, or less than 500 pM.

The term “vector,” as used herein, refers to any molecule (e.g., nucleic acid, plasmid, or virus) that is used to transfer coding information to a host cell. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “expression vectors.”

The term “operably linked,” as used herein, refers to an arrangement of flanking sequences wherein the flanking sequences so described are configured or assembled so as to perform their usual function. Thus, a flanking sequence operably linked to a coding sequence may be capable of effecting the replication, transcription, and/or translation of the coding sequence. For example, a coding sequence is operably linked to a promoter when the promoter is capable of directing transcription of that coding sequence. A flanking sequence need not be contiguous with the coding sequence, so long as it functions correctly.

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the amino acid sequences, proteins, or recombinant polypeptides of the disclosure, including bacterial, yeast, baculoviral, insect and mammalian expression systems (as well as phage display expression systems).

In some embodiments, the disclosure provides methods for preparing an amino acid sequence, protein, or recombinant polypeptide of the disclosure, which methods comprise cultivating or maintaining a host cell under conditions such that the host cell produces or expresses the amino acid sequence, protein, or recombinant polypeptide, and optionally further comprises isolating the amino acid sequence, protein, or recombinant polypeptide so produced.

The term “naturally occurring,” as used herein and applied to a particular molecule, refers to a molecule that is found in nature and has not been manipulated by man. Similarly, the term “non-naturally occurring,” as used herein, refers to a molecule that is not found in nature or that has been structurally modified or synthesized by man.

The terms “recombinant” and “engineered,” as used herein and applied to a particular molecule, such as a polypeptide, refers to a molecule that has been modified or manipulated, such as by mutation, truncation, deletion, substitution, addition, conjugation, or by otherwise changing the primary sequence, chemical or three-dimensional structure, chemical signature, folding behavior, glycosylation state, or any other attribute of the molecule, such that the molecule differs from its naturally occurring counterpart.

The term “patient” as used herein includes human and animal subjects.

A “disorder” is any condition that would benefit from treatment using the amino acid sequences, proteins, or recombinant polypeptides of the disclosure. “Disorder” and “condition” are used interchangeably herein. In particular, a disorder refers to a condition mediated by HERV-K subtype HML-2. For example, a cancer, neurodegenerative disease, or immune disorder expressing HML-2. Types of cancers that can be treated with the recombinant polypeptides as disclosed herein can include, but is not limited to breast, brain, prostate, melanoma, germ cell tumors, ovarian, pancreatic, testes, glioblastoma, teratocarcinoma, medulloblastoma, lung, hepatocellular, colorectal, sarcoma, lymphoma, and/or metastases thereof. Types of neurodegenerative diseases that can be treated with the recombinant polypeptides as disclosed herein can include, but is not limited to ALS, Jacob Creutzfeldt Disease, Alzheimer's disease, and Frontotemporal dementia. Types of immune disorders that can be treated with the recombinant polypeptides as disclosed herein can include, but is not limited to Rheumatoid arthritis, myalgic encephalomyelitis/chronic fatigue syndrome, and Lupus.

As used herein, the terms “treatment,” “treat,” or “treating” refer to a method of reducing the effects of a disease or condition or symptom of the disease or condition. Thus, in the methods disclosed herein, treatment can refer to a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 5% reduction in one or more symptoms of the disease in a subject as compared to a control. Thus, the reduction can be a 5%, 10%, 20%, 30%, 40%, 50%, 60%, 70%, 80%, 90%, 100% or any percent reduction between 5% and 100% as compared to native or control levels. It is understood that treatment does not necessarily refer to a cure or complete ablation of the disease, condition, or symptoms of the disease or condition.

The terms “pharmaceutical composition” or “therapeutic composition,” as used herein, refer to a compound or composition capable of inducing a desired therapeutic effect when properly administered to a patient.

The term “pharmaceutically acceptable carrier” or “physiologically acceptable carrier,” as used herein, refers to one or more formulation materials suitable for accomplishing or enhancing the delivery of the amino acid sequences, proteins, or polypeptides of the disclosure.

The term “therapeutically effective amount” when used in reference to a pharmaceutical composition comprising one or more amino acid sequences, proteins, or recombinant polypeptides of the disclosure refers to an amount or dosage sufficient to produce a desired therapeutic result. More specifically, a therapeutically effective amount is an amount of one or more amino acid sequences, proteins, or recombinant polypeptides of the disclosure sufficient to inhibit, for some period of time, one or more of the clinically defined pathological processes associated with the condition being treated. The therapeutically effective amount may vary depending on the specific amino acid sequence, protein, or recombinant polypeptide that is being used, and also depends on a variety of factors and conditions related to the patient being treated and the severity of the disorder. The determination of a therapeutically effective amount of a given pharmaceutical composition is well within the ability of those of skill in the art.

Therapeutic Compositions Comprising Recombinant Polypeptides that Specifically Bind an Epitope of HERV-K subtype HML-2 and Administration Thereof

In another embodiment, the disclosure provides proteins or recombinant polypeptides comprising or consisting of an amino acid sequence as disclosed herein. In another embodiment, the disclosure provides fusion proteins and multivalent and multispecific fusion proteins comprising or consisting of at least one amino acid sequence, protein, or recombinant polypeptide of the disclosure that is linked to at least one therapeutic agent and/or one or more imaging moieties, optionally via one or more suitable linkers or spacers.

The disclosure further relates to therapeutic uses of the amino acid sequences, proteins, or recombinant polypeptides of the disclosure, or fusion proteins and multivalent and multispecific fusion proteins comprising or consisting of such amino acid sequences, proteins, or recombinant polypeptides, or to pharmaceutical compositions comprising such amino acid sequences, proteins, recombinant polypeptides, fusion proteins, or multivalent and multispecific fusion proteins.

In certain embodiments, the recombinant polypeptide comprise at least one therapeutic agent comprises selected from the group consisting of cytotoxic agents, chemotherapeutic agents, immunotherapeutic agents, radioactive agents, or a biologic agents.

In some embodiments, the therapeutic agent comprises a cytotoxic agent. A cytotoxic agent can be used to deplete cells expressing the HERV-K HML-2 envelope protein.

The cytotoxic agent can exert cytotoxicity when bound to the recombinant polypeptides disclosed herein or can be cleavable and the cytotoxic agent can be cytotoxic when released from the recombinant polypeptide; or the cytotoxic agent can be activated by, for example, electromagnetic radiation. Non-limiting examples of cytotoxic agents can include, but is not limited to anti-microtubule agents, alkylating agents, and DNA minor groove binding agents.

In some embodiments, the therapeutic agent comprises a chemotherapeutic agent selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In some embodiments, the therapeutic agent comprises a radioactive agent selected from 18F, 32P, 33P, 45Ti, 47Sc, 52Fe, 59Fe, 62Cu, 64Cu, 67Cu, 67Ga, 68Ga, 75S, 77As, 86Y, 90Y, 89Sr, 89Zr, 94Tc, 94Tc, 99mTc, 99Mo, 105Pd, 105Rh, 111Ag, 111In, 123I, 124I, 125I, 131I, 142Pr, 143Pr, 149Pm, 153Sm, 154-158Gd, 161Tb, 166Dy, 166Ho, 169Er, 175Lu, 177Lu, 186Re, 188Re, 189Re, 194i, 198Au, 199Au, 211At 211Pb, 212Bi, 212Pb, 213Bi, 223Ra and 225Ac.

In some embodiments, the recombinant polypeptides disclosed herein can be linked to an imaging moiety. Such a combination can be used as reagents for various medical research and diagnostic uses where the HERV-K virus is implicated. For example, imaging moieties can comprise a radiolabel for positron emission tomography (PET) or single photon emission computed tomography (SPECT). The imaging moieties can comprise one or more agents selected from radiolabels, fluorescent labels, enzymatic labels, or PET imaging agents. In certain embodiments, substances that can be used as detectable markers include, but are not limited to ions of transition and lanthanide metals (for example metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

In certain embodiments, the recombinant polypeptides as disclosed here are either directly linked to the at least one therapeutic agent or imaging moiety, or are linked to the at least one therapeutic agent or imaging moiety via a linker or spacer. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids or one (1) amino acid. In certain embodiments, the linker is one amino acid, two amino acids, three amino acids, four amino acids, five amino acids, six amino acids, seven amino acids, eight amino acids, nine amino acids, or ten amino acids.

In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (Ile), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. See e.g., Chen, X. et al., “Fusion Protein Linkers: Property, Design and Functionality” Adv Drug Deliv Rev., 15; 65 (10): 1357-1369 (2013).

In some embodiments, the amino acid composition of a linker can mimic the composition of linkers commonly found in recombinant polypeptides, which can generally by classified as flexible or rigid linkers. For example, flexible linkers found in recombinant polypeptides are generally composed of small, non-polar (e.g., Gly) or polar (e.g., Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser) n, (Gly-Gly-Gly-Ser) n (SEQ ID NO:9), or (Gly-Gly-Gly-Gly-Ser) n (SEQ ID NO:10), where n=1-5. Adjusting the copy number “n” can optimize a linker to achieve sufficient separation of the functional immunogen domains to, e.g., maximize an immunogenic response. Many other flexible linkers have been designed for recombinant polypeptides that can be used herein.

The recombinant polypeptides as disclosed herein can be modified in order to increase their half-life. In an embodiment, recombinant polypeptides comprise at least one amino acid sequence, protein, polypeptide, or other entity to increase the half-life as compared to the unmodified recombinant polypeptide. Such modifications and/or combinations can be prepared and used according to art-recognized methods. Generally, such recombinant polypeptides preferably have a half-life that is at least 1.5 times, or at least 2 times, or at least 5 times, or at least 10 times, or more than 20 times greater than the half-life of the corresponding unmodified recombinant polypeptide. The term “half-life,” as used herein, refers to the time taken for the serum concentration of the recombinant polypeptides of the disclosure to be reduced by 50%, in vivo, as a result, for example, of the degradation of the molecule and/or clearance or sequestration of the recombinant polypeptide by physiological mechanisms. Methods for pharmacokinetic analysis and determination of half-life are familiar to those skilled in the art.

The pharmaceutical compositions disclosed herein can comprise a therapeutically effective amount of recombinant polypeptides as disclosed herein in admixture with a pharmaceutically or physiologically acceptable formulation agent selected for suitability with the mode of administration. Acceptable formulation materials are preferably nontoxic to recipients at the dosages and concentrations to be employed.

Acceptable formulation materials can be used to modify, maintain, or preserve, for example, the pH, osmolarity, viscosity, clarity, color, isotonicity, odor, sterility, stability, rate of dissolution or release, adsorption, or penetration of the composition. Acceptable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides-preferably sodium or potassium chloride- or mannitol sorbitol), delivery vehicles, diluents, excipients and/or pharmaceutical adjuvants (see e.g., Remington's Pharmaceutical Sciences (18th Ed., A. R. Gennaro, ed., Mack Publishing Company 1990), and subsequent editions of the same, which are incorporated herein by reference).

A skilled artisan can determine the optimal pharmaceutical composition comprising the recombinant polypeptides disclosed herein depending upon, for example, the intended route of administration, delivery format, and desired dosage.

The recombinant polypeptides as disclosed herein can be administered in any suitable manner, such as intravenously, via injection or infusion, or in any other suitable manner that allows the recombinant polypeptides to enter the circulation. The preparation of such pharmaceutical compositions is within the knowledge of one of skill in the art.

In some embodiments, the pharmaceutical compositions of the disclosure can also be selected for parenteral delivery. Alternatively, the pharmaceutical compositions can be selected for inhalation or for delivery through the digestive tract, such as orally. The preparation of such pharmaceutical compositions is within the knowledge of one of skill in the art. Additional pharmaceutical compositions will be evident to those of skill in the art, including formulations involving sustained- or controlled-delivery formulations. Techniques for formulating sustained- or controlled-delivery formulations, using, for example, liposome carriers, bio-erodible microparticles or porous beads, and depot injections, are known to those of skill in the art.

In certain embodiments, the disclosure provides a method for preventing and/or treating at least one disease, condition, or disorder mediated by HERV-K subtype HML-2, the method comprising administering to a patient in need thereof a therapeutically or pharmaceutically effective amount of recombinant polypeptides disclosed herein. Types of cancers that can be treated with the recombinant polypeptides as disclosed herein can include, but is not limited to breast, brain, prostate, melanoma, germ cell tumors, ovarian, pancreatic, testes, glioblastoma, teratocarcinoma, medulloblastoma, lung, hepatocellular, colorectal, sarcoma, lymphoma, and/or metastases thereof. Types of neurodegenerative diseases that can be treated with the recombinant polypeptides as disclosed herein can include, but is not limited to ALS, Jacob Creutzfeldt Disease, Alzheimer's disease, and Frontotemporal dementia. Types of immune disorders that can be treated with the recombinant polypeptides as disclosed herein can include, but is not limited to Rheumatoid arthritis, myalgic encephalomyelitis/chronic fatigue syndrome, and Lupus.

The effective amount of a pharmaceutical composition as disclosed herein to be employed therapeutically will depend, for example, upon the therapeutic context and objectives. One of skill in the art will appreciate that an appropriate dosage level for treatment will vary depending, in part, upon the molecule being delivered, the indication for which the composition is being used, the route of administration, and the size (body weight, body surface, or organ size) and condition (age and general health) of the patient.

In certain embodiments, the recombinant polypeptides as disclosed herein can be incorporated into chimeric antigen receptor. The term “chimeric antigen receptor” as used herein is defined as a cell-surface receptor comprising an extracellular binding domain, a transmembrane domain and at least one cytoplasmic signaling domain in a combination that is not naturally found together on a single protein. This particularly includes receptors wherein the extracellular domain and the cytoplasmic domain are not naturally found together on a single receptor protein. Further, the chimeric antigen receptor is different from a T-cell receptor (TCR) expressed in the native T-cell lymphocyte.

The term “CAR T-cells” as used herein refer to a T-cell or population thereof, which has been modified through molecular biological methods to express a chimeric antigen receptor (CAR) on the surface of the T-cell or population of T-cells. The CAR is an engineered polypeptide having an extracellular binding domain with a pre-defined binding specificity to a desired target (i.e., HERV-K subtype HML-2) expressed operably connected to an intracellular part of a T-cell activation domain. The most common CARs are fusions of immunoglobulin binding functionality to transmembrane and cytoplasmic domain (endodomain). Such molecules result in the transmission of a signal in response to recognition by the immunoglobulin binding functionality of its target.

In certain embodiments, a CAR engineered polypeptide comprises: (1) an extracellular binding domain, (2) a transmembrane domain, and (3) at least one cytoplasmic signaling domain.

The extracellular binding domain can also be referred to as an antigen binding domain and can include any domain that will bind to an antigen of interest (i.e., HERV-K subtype HML-2). In certain embodiments, the binding domain contains the recombinant polypeptides disclosed herein, or fragments thereof. In certain embodiments, the recombinant polypeptides include, but are not limited to CDR1, CDR2, CDR3 domains, heavy chains, or fragments thereof. In certain embodiments, the antigen binding domain binds a tumor antigen or tumor associated antigen. In an embodiment, the extracellular binding domain specifically binds an envelope epitope of HERV-K subtype HML-2. In certain embodiments, the extracellular binding domain that specifically binds an envelope epitope of HERV-K subtype HML-2 is a single-domain antibody comprising an amino acid sequence sharing at least 85% sequence identity to the amino acid sequence of SEQ ID NO:3. In an embodiment, the extracellular binding domain comprises the amino acid sequence of SEQ ID NO:3. In an embodiment, the extracellular binding domain consists of the amino acid sequence of SEQ ID NO: 3.

In some embodiments, a CAR comprises a hinge or spacer domain. The hinge domain comprises extracellular structural region of the CAR that separates the extracellular binding domain from the transmembrane domain. In some embodiments, a hinge domain comprises a human IgG-derived CH2 and CH3 regions, for example, the CH2 and CH3 domains of immunoglobulin G1 (IgG1) or IgG4. In certain embodiments, a hinge domain comprises spacers derived from extracellular regions of CD28, CD8a, CD3 or CD4. In an embodiment, the hinge domain is derived from CD28. In another embodiment, the hinge domain is derived from CD8.

A CAR also comprises a transmembrane domain. The transmembrane domain can be any transmembrane domain derived or obtained from any molecule known in the art. The transmembrane domain typically comprises a hydrophobic a helix that spans the cell membrane and primarily serves to anchor the CAR in the T cell membrane. In certain embodiments, the transmembrane domain is fused to the extracellular binding domain of the CAR. The transmembrane domain may be derived from either a natural or synthetic source. In certain embodiments, the transmembrane domain can be derived from any membrane-bound or transmembrane protein. In certain embodiments, the transmembrane is selected from a group including, but not limited to, the alpha, beta, or zeta chain of the T-cell receptor, CD3-epsilon, CD3-zeta, CD4, CD5, CD8, CD9, CD16, CD22, CD28, CD33, CD37, CD45, CD64, CD80, CD86, CD134, CD137, or CD154. In an embodiment, the transmembrane domain is derived from CD28. In another embodiment, the transmembrane domain is derived from CD8.

A CAR also comprises at least one signaling domain, which can also be referred to as the intracellular signaling domain and/or the cytoplasmic co-stimulatory signaling domain of the CAR. The cytoplasmic signaling domain is responsible for activation of at least one of the normal effector functions of the T-cell, and is required for an efficient response of lymphocytes to an antigen. The term “effector function” refers to a specialized function of a cell. Effector function of a T-cell, for example, may be cytolytic activity or helper activity including the secretion of cytokines. Thus, the term “cytoplasmic costimulatory signaling domain” refers to the portion of a protein which transduces the effector function signal and directs the cell to perform a specialized function. While the entire intracellular signaling domain can be employed, in many cases it is not necessary to use the entire chain (i.e., the signaling domain can be derived from the entire protein). To the extent that a truncated portion of the intracellular signaling domain is used, such truncated portion may be used in place of the intact chain as long as it transduces the effector function signal. The term intracellular signaling domain can be derived from and include any truncated portion of the intracellular signaling domain sufficient to transduce the effector function signal. In certain embodiments, the intracellular signaling domain is selected from the cytoplasmic sequences of the T-cell receptor (TCR) and co-receptors that initiate signal transduction following antigen receptor engagement. In certain embodiments, the intracellular signaling domain is selected from a group including, but not limited to, CD2, CD3-zeta, CD3-gamma, CD3-delta, CD3-epsilon, CD5, CD7, CD22, CD27, CD28, CD30, CD40, CD66d, CD79a, CD79b, 4-1BB (CD137), OX40, PD-1, ICOS, lymphocyte function-associated antigen-1 (LFA-1), LIGHT, NKG2C, B7-H3, FcR-gamma, FcR-beta, and TCR-zeta. In an embodiment, the cytoplasmic signaling domain comprises 4-1BB and CD3-zeta. In another embodiment, the cytoplasmic signaling domain comprises CD28 and CD3-zeta.

“Administration” can also encompass in vitro and ex vivo treatments, e.g., of a cell, by a reagent, diagnostic, binding composition, or by another cell. Routes of administration can include, but are not limited to, intravenous administration or infusion techniques. Infusion techniques can involve the administration of the population of activated T-cells through a needle or catheter. Typically, infusion means that the population of activated T-cells is administered intravenously or subcutaneously. In certain embodiments, the population of activated T-cells is administered systemically. In certain embodiments, the population of activated T-cells is administered intravenously (i.e., by intravenous (IV) injection). Preferred routes of administration are intraperitoneally or intravenously.

The present disclosure relates, in part, to the preparation of and use in recipients of CAR T-cell derived effector cells. In certain embodiments, the present disclosure relates to a population of activated T-cells expressing a chimeric antigen receptor (CAR), the CAR comprising an extracellular domain which specifically binds an envelope epitope of HERV-K subtype HML-2.

T-cells used in the methods disclosed herein can be isolated by methods known in the art, including commercially available isolation methods (see, for example, Cartellieri et al., A Novel Ex Vivo Isolation and Expansion Procedure for Chimeric Antigen Receptor Engrafted Human T Cells, 2014; and Ghassemi et al., Reducing Ex Vivo Culture Improves the Antileukemic Activity of Chimeric Antigen Receptor (CAR) T Cells, 2018). Sources for the T-cells include, but are not limited to, peripheral blood, umbilical cord blood, bone marrow, or other sources of hematopoietic cells. Various techniques can be employed to separate the cells to isolate or enrich for desired T-cells. Furthermore, methods for expanding T-cells are well known in the art (see, for example Ghassemi et al., Reducing Ex Vivo Culture Improves the Antileukemic Activity of Chimeric Antigen Receptor (CAR) T Cells, 2018).

The isolated T-cells can be autologous or non-autologous to the subject to which they are administered in the methods of treatment of as disclosed herein. Autologous cells are isolated from the subject to which the population of activated T-cells comprising the CAR are to be administered. In certain embodiment, autologous cells are isolated from the subject to which the isolated and expanded cells recombinantly expressing a CAR are to be administered.

The CAR-T cell compositions described herein can be administered to a subject, either alone or in combination with a pharmaceutically acceptable carrier, in an amount sufficient to induce an appropriate response. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression.

The present disclosure provides methods of generating an immune response in a subject by administering to the subject an effective amount of a CAR T-cell population. An “effective amount” as used herein means an amount which provides a therapeutic or prophylactic benefit. Effective amounts of CAR T-cells can be determined by a physician with consideration of individual differences in age, weight, tumor size, extent of infection or metastasis, and condition of the patient (subject). It can generally be stated that a pharmaceutical composition comprising the population of activated T-cells expressing the CAR as described herein may be administered at a dosage of 10⁴ to 10¹¹ cells/kg body weight, preferably 10⁷ to 10¹⁰ cells/kg body weight, including all integer values within those ranges. T-cell compositions may also be administered multiple times at these dosages. The CAR T-cell population can be administered by any known method, including but not limited to, infusion, regional injection, systemic infusion, intravenously, intracerebroventricularly, intracerebrally, or intratumorally. The cells can be administered by using infusion techniques that are commonly known in immunotherapy. The optimal dosage and treatment regime for a particular patient can readily be determined by one skilled in the art of medicine by monitoring the patient for signs of disease and adjusting the treatment accordingly.

An effective amount of the cell compositions comprising a population of activated T-cells expressing the chimeric antigen receptor as described herein, may be given in one administration of a dose of the population of activated T-cells, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, doses of the population of activated T-cells expressing the chimeric antigen receptor. Where there is more than one administration of a dose, the administration of the doses can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administration of the doses can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, or more, and any combination thereof. The invention is not limited to dosing intervals that are spaced equally in time, but also can encompass doses at non-equal intervals, such as a priming schedule consisting of administration at, for example, 1 day, 4 days, 7 days, and 25 days.

An “effective amount” for a particular subject/patient can vary depending on factors such as the condition or cancer being treated, the overall health of the patient, the route and dose of administration and the severity of side effects. Guidance for methods of treatment and diagnosis is available (see e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). Determination of the number of cells to be administered will be made by one of skill in the art, and will in part be dependent on the extent and severity of cancer, and whether the transfected cells are being administered for treatment of existing cancer or prevention of cancer. The preparation of the pharmaceutical composition containing the activated T-cells will be known to those of skill in the art in light of the present disclosure.

The population of activated T-cells expressing a chimeric antigen receptor of the present disclosure can be administered in a dose, or dosages, where each dose comprises at least 100 cells/kg body weight; at least 1,000 cells/kg body weight; at least 10,000 cells/kg body weight; at least 100,000 cells/kg body weight; at least 1,000,000 cells/kg body weight; at least 10,000,000 cells/kg body weight; at least 100,000,000 cells/kg body weight; at least 1×10⁹ cells/kg body weight; at least 10×10⁹ cells/kg body weight; at least 100×10⁹ cells/kg body weight; or at least 1×10¹² cells/kg body weight.

A dosing schedule of, for example, once/week, twice/week, three times/week, four times/week, five times/week, six times/week, seven times/week, once every two weeks, once every three weeks, once every four weeks, once every five weeks, and the like, can be used. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and up to twelve months or more.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The CAR T-cells, according to the present disclosure, may also be administered with one or more additional therapeutic agents. Methods for co-administration with an additional therapeutic agent are well known in the art (for example, Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). Other agents that can be part of the therapeutic regimen of the subject, such as other immunotherapy, checkpoint inhibitors, immuno-oncology drugs, targeted agents, chemotherapy, and/or radiation.

Without limiting the disclosure, a number of embodiments of the disclosure are described below for purpose of illustration.

The invention will be further described in the following examples, which do not limit the scope of the invention described in the claims.

### Examples

The Examples that follow are illustrative of specific embodiments of the disclosure, and various uses thereof. They are set forth for explanatory purposes only, and should not be construed as limiting the scope of the invention in any way.

### Methods and Materials

Materials: HERV-K Env plasmid was constructed with a synthetic DNA sequence encoding consensus HERV-K Env. The vector used was pcDNA3.1+ from ThermoFisher. A GFP sequence was cloned into the Env plasmid to make the Env-GFP construct. Anti-HERV-K Env monoclonal antibody was from Astral Biologicals. Anti-Myc antibody and magnetic beads were from Sigma. Secondary antibodies were from ThermoFisher. Cell culture media and fetal bovine serum were from ThermoFisher.

Cell transfection: HEK293T cells were seeded onto 6-well plate with a density of 1×10⁶ cells/well. After 24 hours in culture, the cells were transfected with different plasmid using Invitrogen Lipofectamine® 3000 transfection kit. Specifically, 2.5 μg plasmid for each transfection was pre-diluted together with 5 μl P3000 reagent in 125 μl Opti-MEM medium. The diluted plasmid was then mixed with 5 μl Lipofectamine® 3000 which was also pre-diluted in 125 μl Opti-MEM medium. The mixture was incubated at room temperature for 10 minutes and then added drop-wise to the cell culture well. The cells were harvested at 48 hours after transfection.

Immunoprecipitation: The transfected HEK293T cells were washed once with PBS and then lysed in RIPA buffer. The cell lysate was then pre-cleared with anti-Myc magnetic beads for 1 hour at room temperature. After pre-clearance, the cell lysate was incubated with nanobody together with anti-Myc magnetic beads for 2 hours at room temperature. The precipitated beads were washed with RIPA buffer for 3 times and then incubated with 20 μl SDS sample loading buffer at 95° C. for 5 minutes. The supernatant was subjected to Western blot detection.

Western blot: Protein sample in SDS sample loading buffer was separated with 4-20% Bis-Tris PAGE gel and transferred to nitrocellulose membrane. The membrane was first blocked with 5% skim milk in PBST (PBS supplemented with 0.05% Tween-20). It was then incubated with a primary antibody followed by HRP-conjugated secondary antibody. After each antibody incubation, membrane was washed 3 times with PBST. After final wash, the membrane was incubated with ThermoFisher SuperSignal West Pico Plus chemiluminescent reagents and imaged with a chemiluminescence imaging station.

Immunocytochemistry and flow cytometry: Transfected HEK293T cells were dissociated and fixed in suspension with 4% paraformaldehyde in PBS buffer. The cells were then blocked with 5% goat serum in PBS and consecutively incubated with G10, anti-Myc antibody, and secondary antibody. After each incubation, the cells were washed 3 times with PBS. For immunocytochemistry, the secondary antibody was conjugated with Alexa Fluor 596. ProLong™ Gold Antifade Mountant with DAPI. Images were taken with a cell imaging system. For cell cytometry, the secondary antibody was conjugated with Alexa Fluor 647. The stained cells in suspension were analyzed by flow cytometer with GFP and Alexa Fluor 647 channels.

Determination of Binding Interactions with DSF: DSF or protein thermal shift assays were completed by use of an Applied Biosystems QuantStudio 6 Flex real time PCR instrument with 384-well plate or a QuantaBio qPCR instrument. In these reactions, the assay plates contained a final volume of 20 μL with PBS (10 mM phosphate and 150 mM NaCl [pH 7.4]) and protein samples (purified HERV-K Env, 100 nM) plus the binding partner G10 (1 pM-33 nM) and protein thermal shift Dye kit (cat #4461146, diluted 1:125 in kit diluent). The plate was sealed and mixed briefly, followed by plate centrifugation at 1,000 rpm for 2 minutes. After 30-minute incubation at 25° C., the plate was then subjected to thermal shift by ramping the temperature from 25° C. to 99° C. at 0.05° C. increments per second. The relative fluorescence emitted by the thermal shift dye was recorded during the temperature ramp phase and plotted versus temperature. The derivative of these thermal melt curves was determined and Tm calculated from these data. The Tm values were plotted as a function of G10 concentration in GraphPad Prism, and the inflection point of the curve determines the binding affinity of ligand to binding protein. The experiments were completed with n=4 replicate samples per treatment.

### Example 1: Screening and Isolation of Nanobody

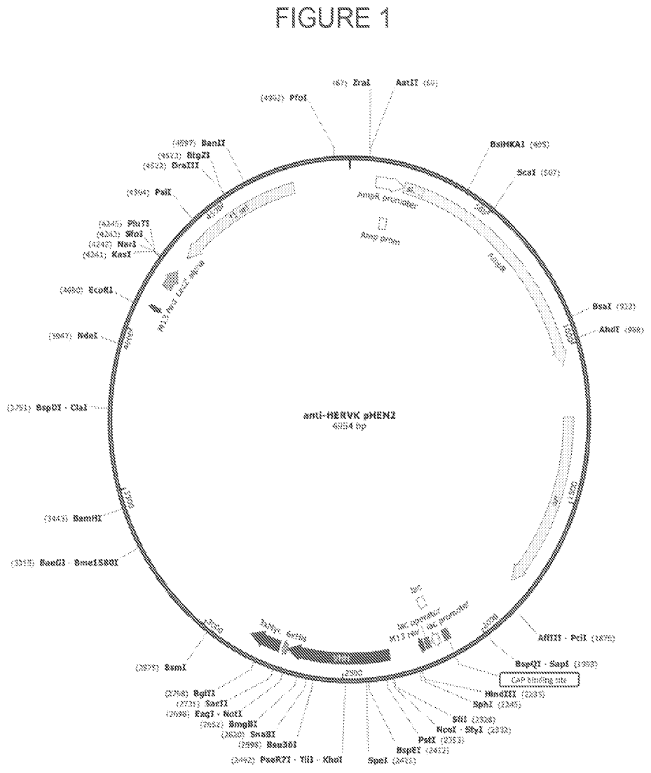

Nanobodies targeted against the human endogenous retrovirus-K (HERV-K) Envelope (Env) were isolated using the Hybrigenics' Antibody Phage Display selection technology according to manufacturer's protocols. The phage display library was screened against cells that expressed the HERV-K Env protein. The screen yielded six nanobody clones termed: A03, B01, G10, A11, F02, and G02. Nanobody clone G10 was selected from these nanobodies since it had the highest affinity of binding as described below. The G10 nanobody construct encodes a 175 amino acids protein (FIG. 1), which includes a 6×His tag for recombinant protein purification with Immobilized metal affinity chromatography, and a 3×Myc tag for nanobody detection with anti-Myc antibody.

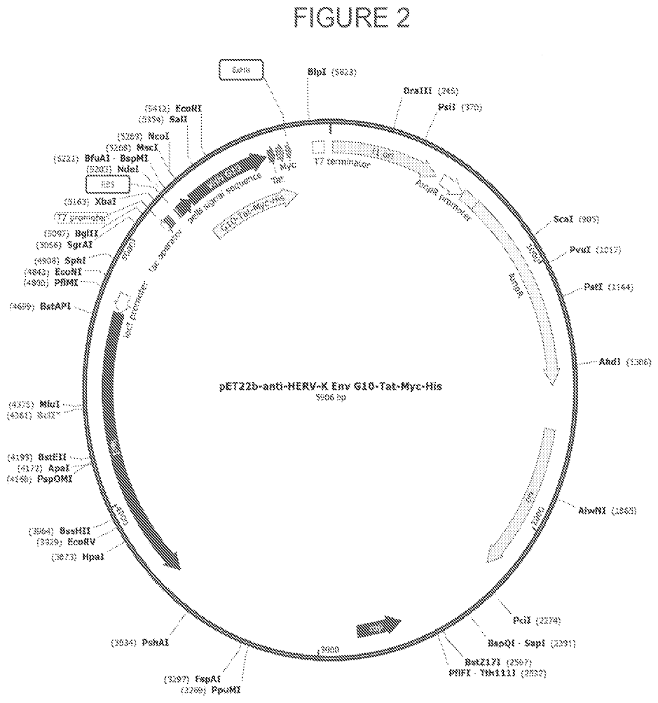

Modification of nanobody to improve cell penetration: A modified version of G10, called G10-Tat, was made in order to improve its cell penetration property. A cell penetrating peptide Tat (amino acid sequence RKKRRQRRR; SEQ ID NO:8) was incorporated into the original construct (FIG. 2), while the tags were re-arranged. The isoelectric point (pl) was changed from 4.6 (G10) to 9.2 (G10-Tat).

>G10 cDNA
(SEQ ID NO: 01)
ATGGCGGAAGTGCAGCTGCAGGCTTCCGGGGGAGGATTTGTGCAGCCGGGGGGGTCATTGCGACTGAG
CTGCGCCGCATCCGGATCAACATGGTATCTGGATGCAATGGGCTGGTTTCGTCAGGCCCCTGGCAAGG
AGAGAGAGTTCGTTTCCGCCATCTCCGACCTGGACGACACAGCACGTTACTACGCTGACAGCGTAAAG
GGAAGATTTACAATTAGCCGGGATAACTCCAAAAACACGGTCTATCTCCAGATGAACAGCCTCAGGGC
CGAGGACACAGCTACGTATTACTGTGCCTATCTGGGTGCAACAATGGCAGAGACATATTGGGGACAGG
GGACGCAGGTAACTGTGAGTAGCGCGGCCGCACATCATCATCACCATCACGGGGCCGCGGAACAAAAA
CTCATCTCAGAAGAGGATCTGAATGGGGCCGCAGAGCAAAAGCTAATATCTGAAGAAGATCTCAACGG
GGCCGCAGAACAGAAACTTATCAGTGAGGAGGACTTGAATGGGGCCGCATAG
>G10-Tat cDNA
(SEQ ID NO: 02)
ATGGCTGAGGTGCAGTTGCAAGCCTCCGGCGGCGGTTTCGTTCAACCTGGAGGTTCTTTGCGTTTGTC
TTGCGCCGCGTCAGGGTCGACTTGGTATTTAGATGCGATGGGATGGTTCCGGCAAGCACCAGGGAAAG
AACGCGAATTCGTTTCAGCAATTAGCGATTTAGACGACACGGCGCGGTACTACGCTGATTCAGTAAAG
GGCCGCTTCACCATTAGTCGTGATAATTCAAAAAATACAGTATATCTTCAGATGAACTCTCTGCGTGC
TGAGGACACCGCAACGTACTACTGCGCTTATCTTGGGGCCACAATGGCAGAGACCTATTGGGGCCAGG
GAACACAAGTCACGGTCAGCAGTGGAGCGGCCCGCAAAAAGAGACGGCAGCGGCGGAGAGGAGCAGCG
GAGCAAAAATTAATTAGCGAAGAGGATCTTAATGGGGCTGCGCATCACCATCATCATCATTAG
>G10 protein
MAEVQLQASGGGFVQPGGSLRLSCAASGSTWYLDAMGWFRQAPGKEREFVSAISDLDDTARYYADSVK
GRFTISRDNSKNTVYLQMNSLRAEDTATYYCAYLGATMAETYWGQGTQVTVSSAAAHHHHHHGAAEQK
LISEEDLNGAAEQKLISEEDINGAAEQKLISEEDINGAA (SEQ ID NO: 03; CDR regions
in bold and underline)
>G10-Tat protein
(SEQ ID NO: 04)
MAEVQLQASGGGFVQPGGSLRLSCAASGSTWYLDAMGWFRQAPGKEREFVSAISDLDDTARYYADSVK
GRFTISRDNSKNTVYLQMNSLRAEDTATYYCAYLGATMAETYWGQGTQVTVSSGAARKKRRQRRRGAA
EQKLISEEDLNGAAHHHHHH
>G10 CDR1
(SEQ ID NO: 05)
STWYLDAMG
>G10 CDR2
(SEQ ID NO: 06)
FVSAISDLDDTARYY
>G10 CDR3
(SEQ ID NO: 07)
YLGATMAETY

### Example 2: Production and Characterization of Recombinant Nanobody

Recombinant nanobodies were made in E. coli by transforming them with each of the six plasmids that encoded the following nanobodies: A03, B01, G10, A11, F02, and G02. Recombinant HERV-K Env nanobodies were isolated and characterized by immunoprecipitation, immunofluorescent staining, and flow cytometry.

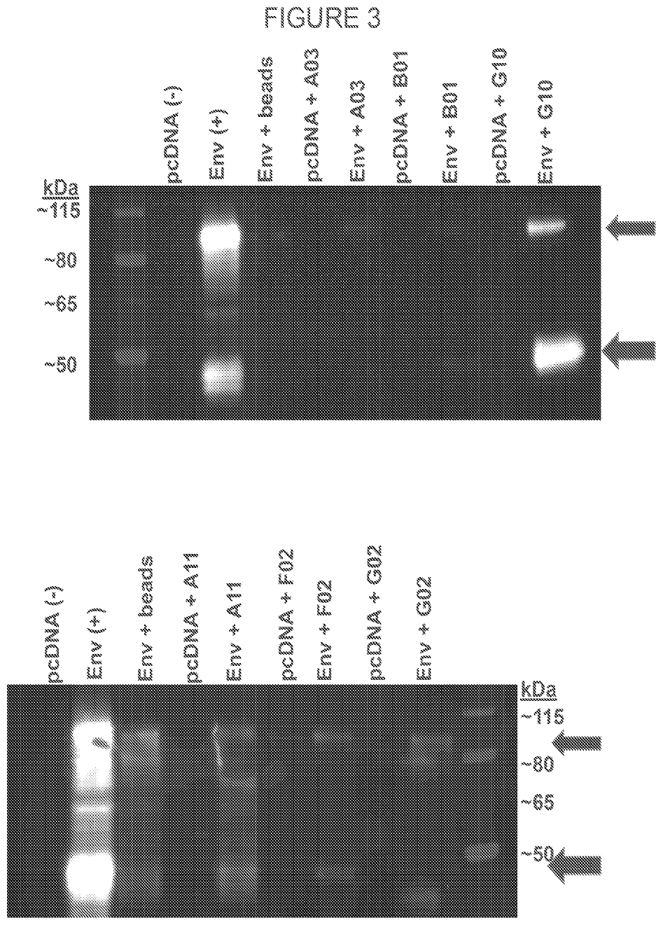

Binding of nanobodies to HERV-K Env by immunoprecipitation: HEK293T cells were transfected with HERV-K Env-Flag plasmid. Cell lysate was harvested after 48 hours of transfection. HERV-K Env was than immunoprecipitated from the cell lysate with the different nanobodies (A03, B01, G10, A11, F02, and G02). The precipitates were then detected by Western blot analysis using an anti-Flag antibody. As seen in FIG. 3, only G10 showed strong binding of both full length and transmembrane subunit of HERV-K Env protein, while other five nanobodies had significantly weaker binding than G10. Because G10 was the most efficient nanobody for HERV-K Env binding, it was picked for the subsequent experiments.

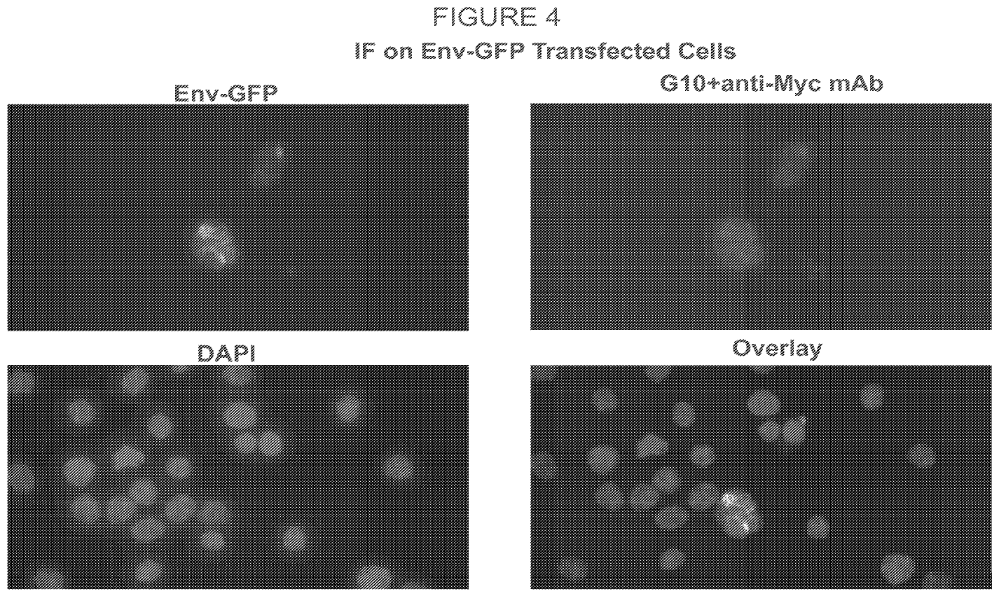

Detection of HERV-K Env by immunostaining with G10: HEK293T cells were transfected with a plasmid encoding Env-GFP fusion protein. After 48 hours of transfection, the cells were fixed and consecutively stained with G10, anti-Myc antibody, and a secondary antibody labeled with Alexa Fluor 596. As seen in FIG. 4, GFP channel showed Env-GFP expression, which was also detected by G10, indicating that G10 is suitable for immunofluorescent staining of Env protein.

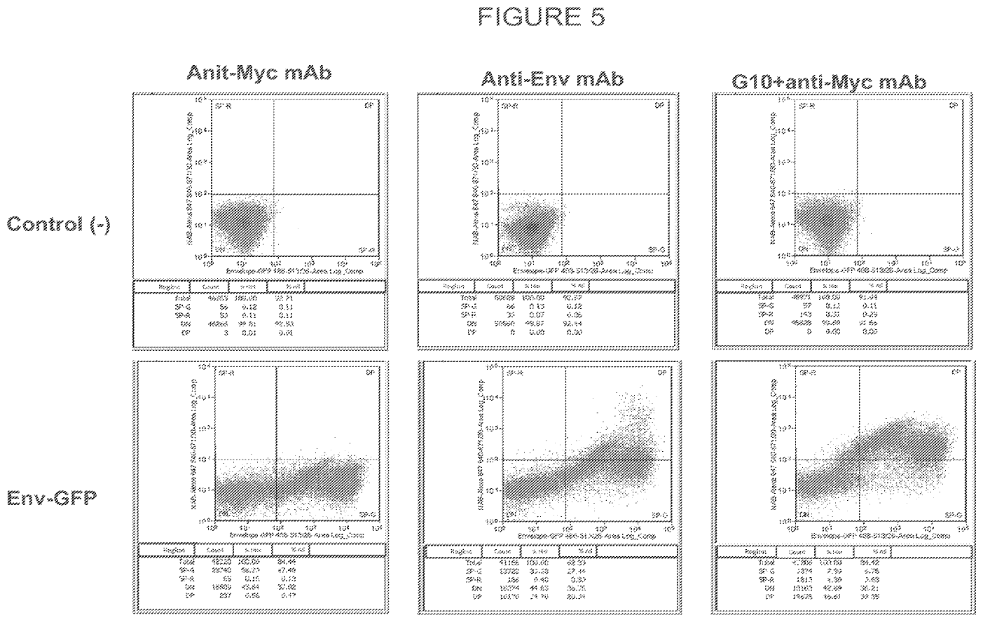

Detection of HERV-K Env by flow cytometry using G10: HEK293T cells were transfected with a plasmid encoding HERV-K Env-green fluorescent protein (GFP) fusion protein. After 48 hours of transfection, the cells were fixed and subsequently stained with G10, followed by anti-Myc antibody, and then a secondary antibody labeled with Alexa Fluor 647. The stained cells in suspension were analyzed by flow cytometry with GFP channel for Env-GFP detection (x-axis) and Alexa Fluor 647 channel for antibody binding of Env (y-axis). As seen in FIG. 5, The G10 staining showed more sensitive detection of Env compared to the commercial antibody against HERV-K Env (comparing the DP quadrant, 39.35% vs 20.34%).

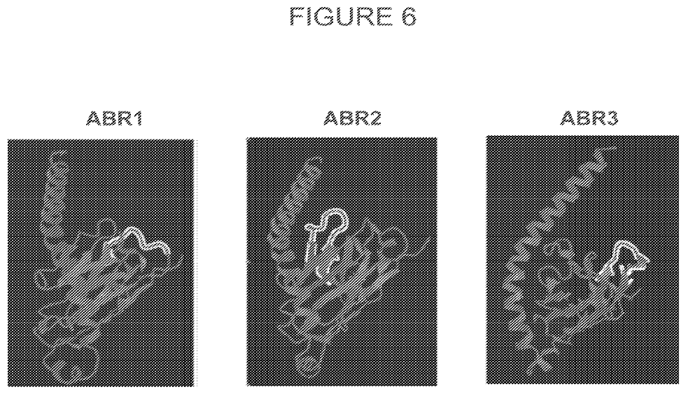

Antigen binding region (ABR) identified: Using Alphafold2, the 3D structure of the G10 clone was modeled with the ABR region highlighted (see FIG. 6).

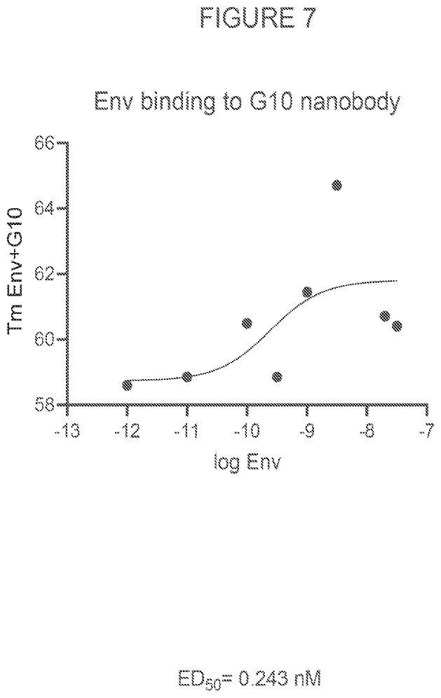

G10 nanobody affinity binding to HERV-K Env: To assess interactions of nanobody G10 with HERV-K envelope protein, we employed differential scanning fluorimetry (DSF) known as protein thermal shift assays. (Ericsson 2006; Niesen 2007; Pantoliano 2001) We determined that PBS at pH 7.4 (physiologic conditions) usage resulted in reproducible protein melting temperatures (Tₘ). Upon incubation of HERV-K envelope with increasing concentrations of G10, we observed a concentration-dependent shift in Tₘ of about 2.5° C. with 1-3 nM G10. The calculated ED₅₀ is 0.243 nm (see FIG. 7).

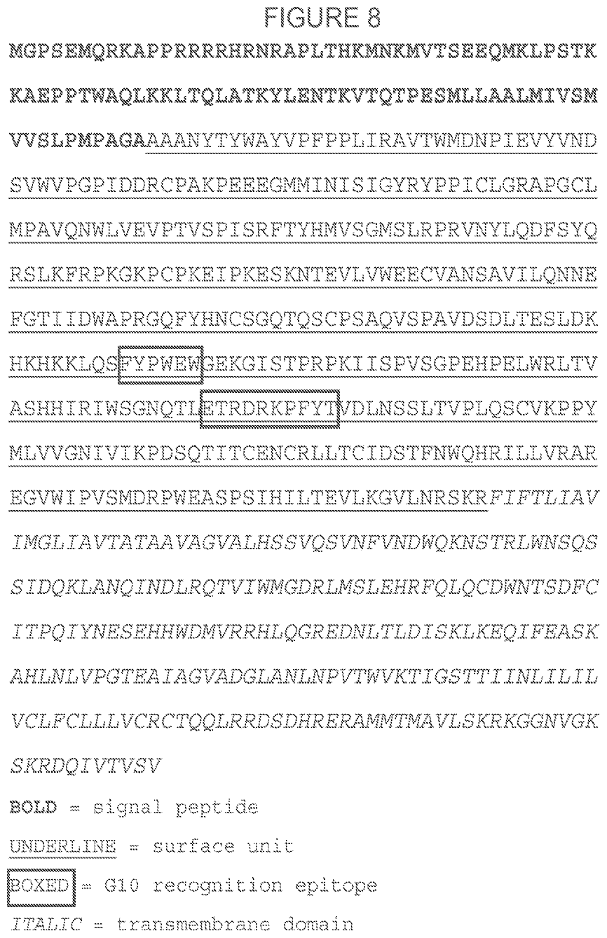

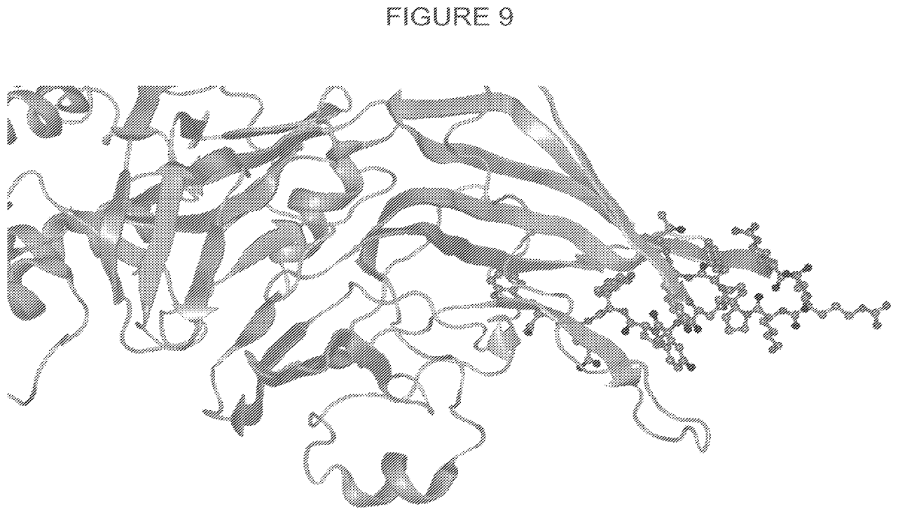

Identification of HERV-K Env epitope: Epitope fingerprinting technique by EPITOPIC GmbH revealed that the likely HERV-K Env epitope for G10 binding consists of 310-FYPWEW (SEQ ID NO:12) and 361-ETRDRKPFYT (SEQ ID NO:13), which are in the surface unit (SU) domain of HERV-K Env (see FIG. 8). Alphafold simulation showed that the epitope (in “ball and stick” model) forms two loops in very close proximity (see FIG. 9).

### Example 3: Production and Characterization of HERV-K Env CAR

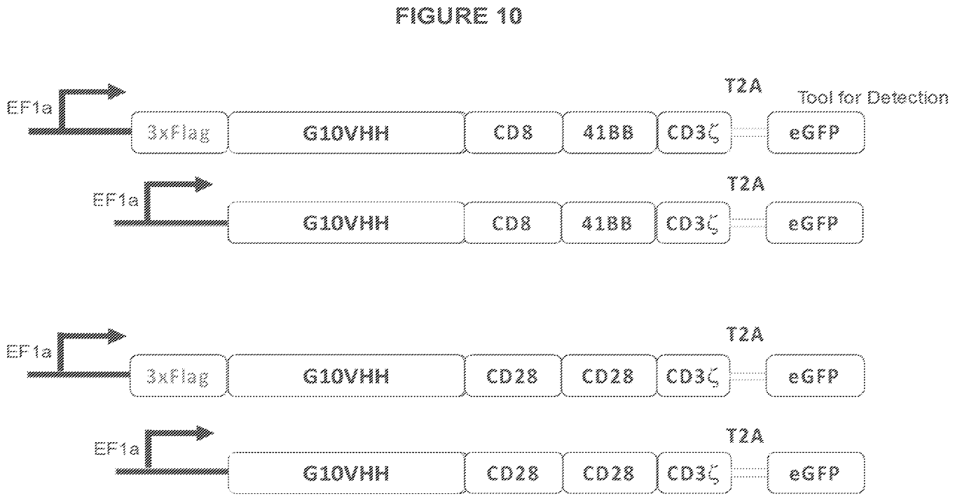

To generate chimeric antigen receptor (CAR) constructs against the HERV-K Envelope glycoprotein (HERV-K Env), the G10 VHH nanobody was incorporated into CAR constructs incorporating either the CD8a hinge and transmembrane domain together with the 4-1BB costimulatory domain or the CD28 hinge and transmembrane domain together with the CD28 costimulatory domain (Hombach et al., 2013). All constructs contain the TCR zeta chain as shown in FIG. 10. To facilitate detection of transduced T cells the eGFP reporter gene was inserted downstream of the CAR construct following a T2A ribosomal skipping sequence (Liu et al., 2017) (FIG. 10). Finally, as cell surface detection reagents for the G10 VHH nanobody CAR are not readily available, 3 copies of the artificial Flag epitope tag were inserted at the N-terminus of the two different G10 VHH nanobody CARs. Note that constructs without the Flag were also generated as the Flag can potentially interfere with CAR function.

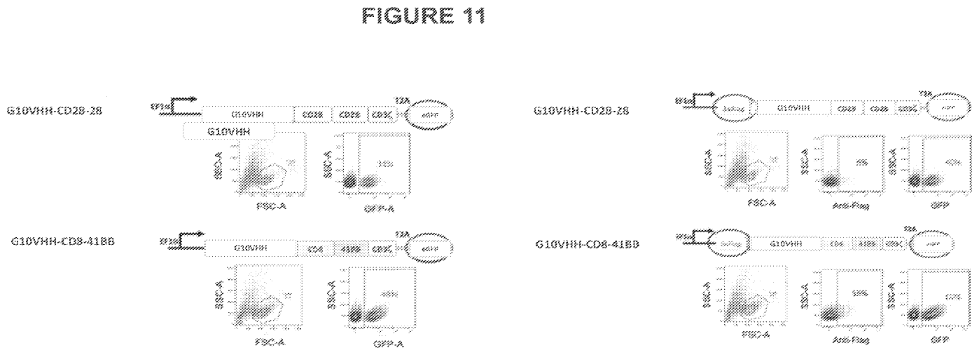

The expression of the different CAR constructs in primary T cells was evaluated following generation of vesicular stomatitis virus Env (VSV)-pseudotyped G10 VHH CAR lentivirus in 293T cells. Briefly, 293T cells were co-transfected with plasmids encoding the G10VHH CAR along with packaging and envelope vectors (pMDLg/pRRE, pMD.2G, and pRSV-Rev). Virus containing supernatants were harvested at 48 h post-transfection and filtered through 0.45 mm-pore size filters. Viral supernatants were concentrated by ultracentrifugation (25,000×g, at 4° C. for 1.5 h) on SW32-Ti rotor through a 20% sucrose cushion. Human T cells were activated with CD3/CD28 monoclonal antibodies (mAb) and transduced at a multiplicity of infection (MOI) of 5 and transduction by all constructs was first assessed by flow cytometry, as a function of GFP levels. As shown in FIG. 11, GFP expression was detected following transduction of T cells with all constructs and expression did not vary markedly between constructs with a CD28 as compared to a 4-1BB costimulatory domain. Notably though, detection of cell surface G10 VHH CAR expression, as a function of binding to an anti-Flag mAb, was significantly lower than overall GFP levels. In a representative transduction, the percentages of Flag⁺ T cells was 4-8-fold lower than the percentages of GFP⁺ T-cells (5% vs 40% and 15% vs 60% for G410 VHH-CD228 and G10 VHH-41BB CARs, respectively; FIG. 11). While the reasons for these differences are not known, the disparate levels may reflect CAR recycling and/or low accessibility of the Flag epitope in the context of the CAR. As such, functional assays were performed by normalizing CAR T-cell numbers on the basis of the percentages of GFP⁺ rather than Flag⁺ cells.

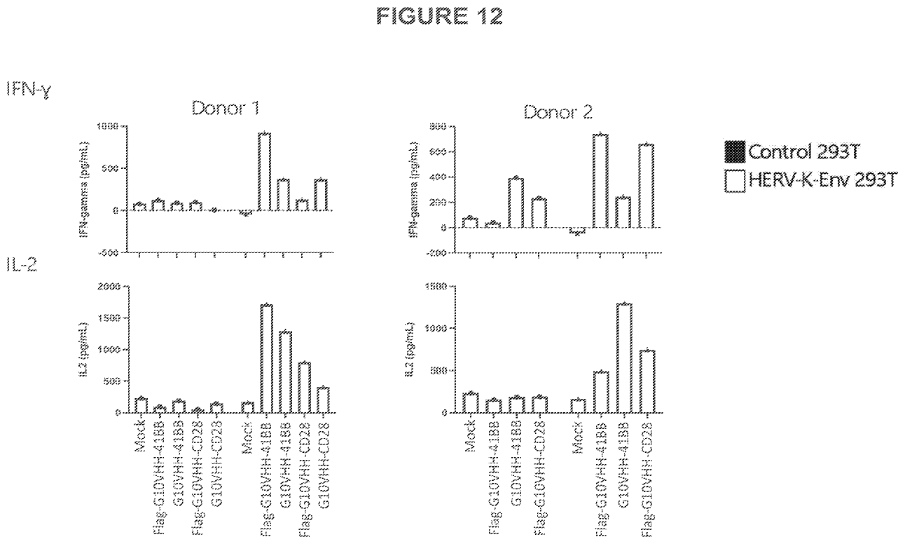

The function of the different G10VHH CAR constructs was studied as a function of cytokine production. The CAR constructs were introduced into primary human T cells using the lentiviral vectors as described above (Qin et al., 2021) and tested in vitro against a HERV-K Env-expressing cell line. To this end the 293T human embryonic kidney cell line was transfected with HERV-K Env and non-transfected 293T were used as a HERV-K Env-negative control. The transduced T cells were co-cultured at a 1:1 effector/target ratio and the production of secreted IFN-γ and IL-2 were monitored by cytokine bead array. Importantly, cytokine secretion of G10 VHH CAR T-cells in response to HERV-K Env-negative targets or by mock-transduced T cells were significantly lower than those detected following coculture with HERV-K Env-positive target cells (FIG. 12). Of note, the level of cytokines secreted by T cells transduced with the different G10 VHH CARs showed donor-to-donor variability in addition to variability between constructs. IL-2 secretion by T cells transduced with all G10 VHH CARs were higher against HERV-K Env+target cells than control cells. Furthermore, all G10 VHH CAR T-cells generated from donor 1 exhibited an increased IFN-γ secretion against HERV-K Env+ target cells (FIG. 12). Together, these data demonstrate ex vivo function of the different G10 VHH CAR constructs against the HERV-K Env target. Additional experiments will further compare these vectors, both in vitro and in vivo, and evaluate the functionality of newly generally G10 VHH CAR constructs designed to optimize CAR-Env epitope interactions.

### Methods and Materials

CAR design: Golden gate assembly was utilized to create each of the indicated CAR constructs as we have previously performed (Chen et al., 2022). All enzymes and reaction buffers were purchased from NEB (Ipswich, MA, USA) and all primers were synthesized by Integrated DNA Technologies (Coralville, IA, USA). The nanobody G10 VHH was used as a template for creating CAR parts in a lentiviral backbone (Dardalhon et al., 2001).

The generated CARs consist of either the CD8 or the CD28 hinge domain, followed by the 4-1BB or the CD28 co-stimulatory domain, respectively. Constructs with a 3×Flag sequence at the N-terminus of the CAR were also generated. The GFP reporter gene was expressed in all vectors downstream of the CAR sequence and a T2A ribosomal skipping motif.

>G10 VHH CD28 T2A eGFP:
(SEQ ID NO: 14)
atggagaccgacaccctgctgctatgggtactgctgctttgggtgcccggaagcaccggtagtatggc
ggaagtgcagctgcaggctTCTGGAggaggatttgtgcagccgGGAgggAGTttgcgactgagctgcg
ccgcatccggatcaacatggtatctggatgcaatgggctggtttcgtcagGCAcctggcaaggagaga
gagttcgtttccgccatctccgacCTCgacgacacagcacgttactacgctgacagcgtaaagggaag
atttacaattagccgggataactccaaaaacacggtctatctccagatgaacagcctcagggccgagg
acacagctacgtattactgtgcctatctgggtgcaacaatggcagagacatattggggacaggggacg
caggtaactgtgagtagctccatcgaagtgatgtacccccctccctacctggataacgagaagagcaa
cggcaccatcatccacgtgaagggaaagcacctgtgtcccagccccctgtttcccggccctagcaagc
ccttctgggtgctggtggtggtcggcggagtgctggcctgctacagcctcctggtgaccgtggccttc
atcatcttctgggtgaggagcaagaggtccaggctgctgcacagcgactacatgaatatgacccccag
aaggcccggccccaccagaaagcactatcagccctacgccccccccagggactttgccgcctacagga
gcagggtgaagttcagcagatccgccgatgcccctgcttaccagcagggccagaaccagctgtataac
gagctgaacctgggcaggagggaggaatacgacgtgctggataagaggaggggaagggaccccgagat
gggcggaaagcccaggaggaagaacccccaggagggcctgtacaatgagctgcagaaagacaagatgg
ccgaggcctacagcgagatcggcatgaagggcgagaggaggaggggcaagggccatgacggcctgtac
caaggcctgtccaccgccaccaaggatacctacgacgccctgcacatgcaggccctgcctcccaggGG
Atccggcgagggcaggggaagtcttctaacatgcggggacgtggaggaaaatcccggcccaatggtga
gcaagGGAgaggagctgttcaccggggtggtgcccatcctggtcgagctggacggcgacgtaaacggc
cacaagttcagcgtgtccGGAgagGGTgagggcgatGCAaccTATggcAAActgaccctgaagttcat
ctgcaccaccggcaagctgcccgtgccctggcccaccctcgtgaccaccctgacctacggcgtgcagt
gcttcagccgctaccccgaccacatgaagcagcacgacttcttcaagtccGCAatgcccgaaggctac
gtccaggagcgcaccatcttcTTTaaggacgacggcaacTATaagacccgcGCAgaggtgaagttcga
gggcgacaccctggtgaaccgcatcgagctgaagGGAatcgacttcaaggagGATGGTaacatcctgG
GAcacaagctggagtacaactacaacagccacaacgtctatatcatgGCAgacaagcagaagaacGGA
atcaaggtgaacttcaagatccgccacaacatcgaggacggcagcgtgcagctcgccgacCATtacca
gcagaacacccccatcGGTgacggccccgtgctgctgcccgacaaccactacctgagcacccagtccG
CActgagcaaagaccccaacgagaagcgcgatcacatggtcctgctggagttcgtgaccgccgccggg
atcactctcGGAatggacgagctgtacaagTGA
>Flag G10 VHH CD28 T2A eGFP:
(SEQ ID NO: 15)
atggagaccgacaccctgctgctatgggtactgctgctttgggtgcccggaagcaccGACTACAAAGA
CCACGACGGCGACTACAAGGACCATGACATCGATTATAAGGATGATGATGATAAAggtagtatggcgg
aagtgcagctgcaggctTCTGGAggaggatttgtgcagccgGGAgggAGTttgcgactgagctgcgcc
gcatccggatcaacatggtatctggatgcaatgggctggtttcgtcagGCAcctggcaaggagagaga
gttcgtttccgccatctccgacCTCgacgacacagcacgttactacgctgacagcgtaaagggaagat
ttacaattagccgggataactccaaaaacacggtctatctccagatgaacagcctcagggccgaggac
acagctacgtattactgtgcctatctgggtgcaacaatggcagagacatattggggacaggggacgca
ggtaactgtgagtagctccatcgaagtgatgtacccccctccctacctggataacgagaagagcaacg
gcaccatcatccacgtgaagggaaagcacctgtgtcccagccccctgtttcccggccctagcaagccc
ttctgggtgctggtggtggtcggcggagtgctggcctgctacagcctcctggtgaccgtggccttcat
catcttctgggtgaggagcaagaggtccaggctgctgcacagcgactacatgaatatgacccccagaa
ggcccggccccaccagaaagcactatcagccctacgccccccccagggactttgccgcctacaggagc
agggtgaagttcagcagatccgccgatgcccctgcttaccagcagggccagaaccagctgtataacga
gctgaacctgggcaggagggaggaatacgacgtgctggataagaggaggggaagggaccccgagatgg
gcggaaagcccaggaggaagaacccccaggagggcctgtacaatgagctgcagaaagacaagatggcc
gaggcctacagcgagatcggcatgaagggcgagaggaggaggggcaagggccatgacggcctgtacca
aggcctgtccaccgccaccaaggatacctacgacgccctgcacatgcaggccctgcctcccaggGGAt
ccggcgagggcaggggaagtcttctaacatgcggggacgtggaggaaaatcccggcccaatggtgagc
aagGGAgaggagctgttcaccggggtggtgcccatcctggtcgagctggacggcgacgtaaacggcca
caagttcagcgtgtccGGAgagGGTgagggcgatGCAaccTATggcAAActgaccctgaagttcatct
gcaccaccggcaagctgcccgtgccctggcccaccctcgtgaccaccctgacctacggcgtgcagtgc
ttcagccgctaccccgaccacatgaagcagcacgacttcttcaagtccGCAatgcccgaaggctacgt
ccaggagcgcaccatcttcTTTaaggacgacggcaacTATaagacccgcGCAgaggtgaagttcgagg
gcgacaccctggtgaaccgcatcgagctgaagGGAatcgacttcaaggagGATGGTaacatcctgGGA
cacaagctggagtacaactacaacagccacaacgtctatatcatgGCAgacaagcagaagaacGGAat
caaggtgaacttcaagatccgccacaacatcgaggacggcagcgtgcagctcgccgacCATtaccagc
agaacacccccatcGGTgacggccccgtgctgctgcccgacaaccactacctgagcacccagtccGCA
ctgagcaaagaccccaacgagaagcgcgatcacatggtcctgctggagttcgtgaccgccgccgggat
cactctcGGAatggacgagctgtacaagTGA
>G10 VHH CD8 4-1BB T2A eGFP
(SEQ ID NO: 16)
atggagaccgacaccctgctgctatgggtactgctgctttgggtgcccggaagcaccggcggtagtat
ggcggaagtgcagctgcaggctTCTGGAggaggatttgtgcagccgGGAgggAGTttgcgactgagct
gcgccgcatccggatcaacatggtatctggatgcaatgggctggtttcgtcagGCAcctggcaaggag
agagagttcgtttccgccatctccgacCTCgacgacacagcacgttactacgctgacagcgtaaaggg
aagatttacaattagccgggataactccaaaaacacggtctatctccagatgaacagcctcagggccg
aggacacagctacgtattactgtgcctatctgggtgcaacaatggcagagacatattggggacagggg
acgcaggtaactgtgagtagctccaccacgacgccagcgccgcgaccaccaacaccggcgcccaccat
cgcgtcgcagcccctgtccctgcgcccagaggcgtgccggccagcggcggggggcgcagtgcacacga
gggggctggacttcgcctgtgatatctacatctgggcgcccttggccgggacttgtggggtccttctc
ctgtcactggttatcaccctttactgcaaacggggcagaaagaaactcctgtatatattcaaacaacc
atttatgagaccagtacaaactactcaagaggaagatggctgtagctgccgatttccagaagaagaag
aaggaggatgtgaactgagggtgaagttcagcagatccgccgatgcccctgcttaccagcagggccag
aaccagctgtataacgagctgaacctgggcaggagggaggaatacgacgtgctggataagaggagggg
aagggaccccgagatgggcggaaagcccaggaggaagaacccccaggagggcctgtacaatgagctgc
agaaagacaagatggccgaggcctacagcgagatcggcatgaagggcgagaggaggaggggcaagggc
catgacggcctgtaccaaggcctgtccaccgccaccaaggatacctacgacgccctgcacatgcaggc
cctgcctcccaggGGAtccggcgagggcaggggaagtcttctaacatgcggggacgtggaggaaaatc
ccggcccaatggtgagcaagGGAgaggagctgttcaccggggtggtgcccatcctggtcgagctggac
ggcgacgtaaacggccacaagttcagcgtgtccGGAgagGGTgagggcgatGCAaccTATggcAAAct
gaccctgaagttcatctgcaccaccggcaagctgcccgtgccctggcccaccctcgtgaccaccctga
cctacggcgtgcagtgcttcagccgctaccccgaccacatgaagcagcacgacttcttcaagtccGCA
atgcccgaaggctacgtccaggagcgcaccatcttcTTTaaggacgacggcaacTATaagacccgcGC
AgaggtgaagttcgagggcgacaccctggtgaaccgcatcgagctgaagGGAatcgacttcaaggagG
ATGGTaacatcctgGGAcacaagctggagtacaactacaacagccacaacgtctatatcatgGCAgac
aagcagaagaacGGAatcaaggtgaacttcaagatccgccacaacatcgaggacggcagcgtgcagct
cgccgacCATtaccagcagaacacccccatcGGTgacggccccgtgctgctgcccgacaaccactacc
tgagcacccagtccGCActgagcaaagaccccaacgagaagcgcgatcacatggtcctgctggagttc
gtgaccgccgccgggatcactctcGGAatggacgagctgtacaagTGA
>Flag G10 VHH CD8 4-1BB T2A eGFP:
(SEQ ID NO: 17)
atggagaccgacaccctgctgctatgggtactgctgctttgggtgcccggaagcaccGACTACAAAGA
CCACGACGGCGACTACAAGGACCATGACATCGATTATAAGGATGATGATGATAAAggtagtatggcgg
aagtgcagctgcaggctTCTGGAggaggatttgtgcagccgGGAgggAGTttgcgactgagctgcgcc
gcatccggatcaacatggtatctggatgcaatgggctggtttcgtcagGCAcctggcaaggagagaga
gttcgtttccgccatctccgacCTCgacgacacagcacgttactacgctgacagcgtaaagggaagat
ttacaattagccgggataactccaaaaacacggtctatctccagatgaacagcctcagggccgaggac
acagctacgtattactgtgcctatctgggtgcaacaatggcagagacatattggggacaggggacgca
ggtaactgtgagtagctccaccacgacgccagcgccgcgaccaccaacaccggcgcccaccatcgcgt
cgcagcccctgtccctgcgcccagaggcgtgccggccagcggggggggcgcagtgcacacgaggggg
ctggacttcgcctgtgatatctacatctgggcgcccttggccgggacttgtggggtccttctcctgtc
actggttatcaccctttactgcaaacggggcagaaagaaactcctgtatatattcaaacaaccattta
tgagaccagtacaaactactcaagaggaagatggctgtagctgccgatttccagaagaagaagaagga
ggatgtgaactgagggtgaagttcagcagatccgccgatgcccctgcttaccagcagggccagaacca
gctgtataacgagctgaacctgggcaggagggaggaatacgacgtgctggataagaggaggggaaggg
accccgagatgggcggaaagcccaggaggaagaacccccaggagggcctgtacaatgagctgcagaaa
gacaagatggccgaggcctacagcgagatcggcatgaagggcgagaggaggaggggcaagggccatga
cggcctgtaccaaggcctgtccaccgccaccaaggatacctacgacgccctgcacatgcaggccctgc
ctcccaggGGAtccggcgagggcaggggaagtcttctaacatgcggggacgtggaggaaaatcccggc
ccaatggtgagcaagGGAgaggagctgttcaccggggggtgcccatcctggtcgagctggacggcga
cgtaaacggccacaagttcagcgtgtccGGAgagGGTgagggcgatGCAaccTATggcAAActgaccc
tgaagttcatctgcaccaccggcaagctgcccgtgccctggcccaccctcgtgaccaccctgacctac
ggcgtgcagtgcttcagccgctaccccgaccacatgaagcagcacgacttcttcaagtccGCAatgcc
cgaaggctacgtccaggagcgcaccatcttcTTTaaggacgacggcaacTATaagacccgcGCAgagg
tgaagttcgagggcgacaccctggtgaaccgcatcgagctgaagGGAatcgacttcaaggagGATGGT
aacatcctgGGAcacaagctggagtacaactacaacagccacaacgtctatatcatgGCAgacaagca
gaagaacGGAatcaaggtgaacttcaagatccgccacaacatcgaggacggcagcgtgcagctcgccg
acCATtaccagcagaacacccccatcGGTgacggccccgtgctgctgcccgacaaccactacctgagc
acccagtccGCActgagcaaagaccccaacgagaagcgcgatcacatggtcctgctggagttcgtgac
cgccgccgggatcactctcGGAatggacgagctgtacaagTGA

The backbone for the modular CARs was created via NheI and SalI double digestion, run on an agarose gel, and recovered using the Zymoclean Gel DNA Recovery Kit (Zymo Research, Irvine, CA, USA). Primers containing BsmBI restriction sites with programmable overhangs specifying orientation and position were used for PCR generation of CAR fragments. Residual template plasmid was removed via DpnI digestion and plasmid was isolated using the QIAquick PCR Purification Kit (Qiagen, Hilden, Germany). One-pot reactions for each construct, consisting of DNA template, modular regions, BsmBI-v2, T4 ligase, and T4 ligase buffer, were performed in a Pro Flex PCR system (Thermo Fisher, Waltham, MA, USA) as described by the manufacturer (Kucera and Cantor, 2018). The resulting reaction mixture was used to transform One Shot™ Stbl3™ Chemically Competent E. coli (ThermoFisher). Single colonies were picked from LB agar plates, cultured overnight, and plasmid DNA was purified using the ZR Plasmid Miniprep-Classic (Zymo Research). Constructs were sequence verified by Sanger sequencing (Psomagen, Rockville, MD, USA) and DNA generated using the NucleoBond Xtra Midi kit for transfection-grade plasmid DNA (Macherey-Nagel, Düren, Germany).

Lentivirus production and generation of CART cells: CAR-encoding lentiviral vectors were produced by transient transfection of a HEK 293T cell line as previously described (Shalabi et al., 2022). Briefly, 293T cells were plated into poly-D lysine-coated 15-cm plates (BD Biosciences). The following day, 293T cells were co-transfected using LIPOFECTAMINE™ 3000 (Life Technologies) with plasmids encoding the G10 VHH CAR along with packaging and envelope vectors (pMDLg/pRRE, pMD.2G, and pRSV-Rev). Virus containing supernatants were harvested at 48 hours post-transfection and filtered through 0.45 mm-pore size filters. Viral supernatants were concentrated by ultracentrifugation (90,000×g, at 4° C. for 1.5 h) on SW32-Ti rotor through a 20% sucrose cushion.

Human peripheral blood mononuclear cells from normal donors were activated with a 1:1 ratio of CD3/CD28 microbeads (Dynabeads Human T-Expander, Life Technologies) in AIM-V media containing 40 IU/mL recombinant IL-2 (teceleukin, rhIL-2; Roche) for 24 hours. Activated T-cells (2e6) were added to 6-well plates in a final volume of 5 mL of AIM-V media together with 10 μg/mL protamine sulfate, 100 IU/mL IL-2, and lentiviral vectors to achieve an MOI of 5.

Plates were centrifuged at 1000 g for 2 hours at 32° C. and then incubated at 37° C. overnight. On day 3 post-transduction, CD3/CD28 beads were removed and cells were cultured at a concentration of 500,000 cells/mL in fresh AIM-V media containing 100 IU/mL IL-2. Fresh IL2-containing media was added every 2 to 3 days until harvest at day 9.

Depending on the CAR construct, CAR surface expression was assessed using a mouse anti-FLAG M2 antibody (F3165 Sigma-Aldrich) and/or as a function of GFP expression. Data were acquired on a Fortessa FACS cytometer (BD Biosciences) and analyzed using FlowJo software (V10, Treestar, Ashland, OR).

Cytokine secretion assay: CAR T-cells, evaluated as a function of GFP expression (5e4 cells), or control T-cells were washed 3 times and then co-cultured at a 1:1 ratio with HERV-K Env-negative or -positive HEK 293T target cells in 48-well plates. HEK 293T cells expressing the HERV-K Env antigen were generated by transfection with a pcDNA3.1-HERV-K Env plasmid (2 μg). Cytokine levels in culture supernatants were measured using IFNg and IL-2 enzyme-linked immunosorbent assay kits (BD Biosciences) as per the manufacturer's instructions.

### REFERENCES

1. Wang S S, Yan Y S, Ho K. 2021. U S FDA-approved therapeutic antibodies with high-concentration formulation: summaries and perspectives. Antib Ther 4:262-272.
2. Liu J K. 2014. The history of monoclonal antibody development-Progress, remaining challenges and future innovations. Ann Med Surg (Lond) 3:113-6.
3. Hamers-Casterman C, Atarhouch T, Muyldermans S, Robinson G, Hamers C, Songa E B, Bendahman N, Hamers R. 1993. Naturally occurring antibodies devoid of light chains. Nature 363:446-8.
4. Muruganandam A, Tanha J, Narang S, Stanimirovic D. 2002. Selection of phage-displayed llama single-domain antibodies that transmigrate across human blood-brain barrier endothelium. FASEB J 16:240-2.
5. Yang E Y, Shah K. 2020. Nanobodies: Next Generation of Cancer Diagnostics and Therapeutics. Front Oncol 10:1182.
6. Garcia-Montojo M, Doucet-O'Hare T, Henderson L, Nath A. 2018. Human endogenous retrovirus-K (HML-2): a comprehensive review. Crit Rev Microbiol 44:715-738.
7. Li W, Lee M H, Henderson L, Tyagi R, Bachani M, Steiner J, Campanac E, Hoffman D A, von Geldern G, Johnson K, Maric D, Morris H D, Lentz M, Pak K, Mammen A, Ostrow L, Rothstein J, Nath A. 2015. Human endogenous retrovirus-K contributes to motor neuron disease. Sci Transl Med 7: 307ra153.
8. Herbst H, Sauter M, Mueller-Lantzsch N. 1996. Expression of human endogenous retrovirus K elements in germ cell and trophoblastic tumors. Am J Pathol 149:1727-35.
9. Muster T, Waltenberger A, Grassauer A, Hirschl S, Caucig P, Romirer I, Fodinger D, Seppele H, Schanab O, Magin-Lachmann C, Lower R, Jansen B, Pehamberger H, Wolff K. 2003. An endogenous retrovirus derived from human melanoma cells. Cancer Res 63:8735-41.
10. Buscher K, Trefzer U, Hofmann M, Sterry W, Kurth R, Denner J. 2005. Expression of human endogenous retrovirus K in melanomas and melanoma cell lines. Cancer Res 65:4172-80.
11. Wang-Johanning F, Liu J, Rycaj K, Huang M, Tsai K, Rosen D G, Chen D T, Lu D W, Barnhart K F, Johanning G L. 2007. Expression of multiple human endogenous retrovirus surface envelope proteins in ovarian cancer. Int J Cancer 120:81-90.
12. Oricchio E, Sciamanna I, Beraldi R, Tolstonog G V, Schumann G G, Spadafora C. 2007. Distinct roles for LINE-1 and HERV-K retroelements in cell proliferation, differentiation and tumor progression. Oncogene 26:4226-33.
13. Schmitt K, Reichrath J, Roesch A, Meese E, Mayer J. 2013. Transcriptional profiling of human endogenous retrovirus group HERV-K (HML-2) loci in melanoma. Genome Biol Evol 5:307-28.
14. Bhardwaj N, Montesion M, Roy F, Coffin J M. 2015. Differential expression of HERV-K (HML-2) proviruses in cells and virions of the teratocarcinoma cell line Tera-1. Viruses 7:939-68.
15. Li M, Radvanyi L, Yin B, Rycaj K, Li J, Chivukula R, Lin K, Lu Y, Shen J, Chang D Z, Li D, Johanning G L, Wang-Johanning F. 2017. Downregulation of Human Endogenous Retrovirus Type K (HERV-K) Viral env RNA in Pancreatic Cancer Cells Decreases Cell Proliferation and Tumor Growth. Clin Cancer Res 23:5892-5911.
16. Zhou F, Li M, Wei Y, Lin K, Lu Y, Shen J, Johanning G L, Wang-Johanning F. 2016. Activation of HERV-K Env protein is essential for tumorigenesis and metastasis of breast cancer cells. Oncotarget 7:84093-84117.
17. Doucet-O'Hare T T, DiSanza B L, DeMarino C, Atkinson A L, Rosenblum J S, Henderson L J, Johnson K R, Kowalak J, Garcia-Montojo M, Allen S J, Orr B A, Santi M, Wang T, Fathi S, Lee M H, Sampson K, Li W, Zhuang Z, Nath A. 2021. SMARCB1 deletion in atypical teratoid rhabdoid tumors results in human endogenous retrovirus K (HML-2) expression. Sci Rep 11:12893.
18. Ericsson U. B., Hallberg B. M., Detitta G. T., Dekker N., Nordlund P. Thermofluor-based high-throughput stability optimization of proteins for structural studies. Anal. Biochem. 2006; 357:289-298.
19. Niesen F. H., Berglund H., Vedadi M. The use of differential scanning fluorimetry to detect ligand interactions that promote protein stability. e2007; 2:2212-2221.
20. Pantoliano M. W., Petrella E. C., Kwasnoski J. D., Lobanov V. S., Myslik J., Graf E., Carver T., Asel E., Springer B. A., Lane P., Salemme F. R. High-density miniaturized thermal shift assays as a general strategy for drug discovery. J. Biomol. Screen. 2001; e: 429-440.
21. Chen, X., Mirazee, J. M., Skorupka, K. A., Matsuo, H., Youkharibache, P., Taylor, N., and Walters, K. J. (2022). The CD8alpha hinge is intrinsically disordered with a dynamic exchange that includes proline cis-trans isomerization. J Magn Reson 340, 107234. 10.1016/j.jmr.2022.107234.
22. Dardalhon, V., Herpers, B., Noraz, N., Pflumio, F., Guetard, D., Leveau, C., Dubart-Kupperschmitt, A., Charneau, P., and Taylor, N. (2001). Lentivirus-mediated gene transfer in primary T cells is enhanced by a central DNA flap. Gene Ther 8, 190-198.
23. Hombach, A. A., Holzinger, A., and Abken, H. (2013). The weal and woe of costimulation in the adoptive therapy of cancer with chimeric antigen receptor (CAR)-redirected T cells. Curr Mol Med 13, 1079-1088.
24. Liu, Z., Chen, O., Wall, J. B. J., Zheng, M., Zhou, Y., Wang, L., Vaseghi, H. R., Qian, L., and Liu, J. (2017). Systematic comparison of 2A peptides for cloning multi-genes in a polycistronic vector. Scientific reports 7, 2193. 10.1038/s41598-017-02460-2.
25. Qin, H., Yang, L., Chukinas, J. A., Shah, N., Tarun, S., Pouzolles, M., Chien, C. D., Niswander, L. M., Welch, A. R., Taylor, N., et al. (2021). Systematic preclinical evaluation of CD33-directed chimeric antigen receptor T cell immunotherapy for acute myeloid leukemia defines optimized construct design. J Immunother Cancer 9. 10.1136/jitc-2021-003149.
26. Shalabi, H., Qin, H., Su, A., Yates, B., Wolters, P. L., Steinberg, S. M., Ligon, J. A., Silbert, S., DeDe, K., Benzaoui, M., et al. (2022). CD19/22 CAR T cells in children and young adults with B-ALL: phase 1 results and development of a novel bicistronic CAR. Blood 140, 451-463. 10.1182/blood.2022015795.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A recombinant polypeptide that specifically binds to an envelope epitope of human endogenous retrovirus K (HERV-K) subtype HML-2, wherein the polypeptide comprises:
(a) the amino acid sequence of SEQ ID NOs: 3 or 4, or a fragment thereof;
(b) an amino acid sequence that is at least 85% identical to the amino acid sequence of SEQ ID NOs: 3 or 4; or
(c) complementarity determining regions, CDR1, CDR2, and CDR3, wherein:
(i) CDR1 comprises the amino acid sequence of SEQ ID NO:5, or an amino acid sequence that is at least 85% identical to the amino acid sequence of SEQ ID NO:5;
(ii) CDR2 comprises the amino acid sequence of SEQ ID NO:6, or an amino acid sequence that is at least 85% identical to the amino acid sequence of SEQ ID NO:6; and
(iii) CDR3 comprises the amino acid sequence of SEQ ID NO:7, or an amino acid sequence that is at least 85% identical to the amino acid sequence of SEQ ID NO:7.

2. The recombinant polypeptide of claim 1, wherein the recombinant polypeptide comprises the amino acid sequence of SEQ ID NO:3.

3. The recombinant polypeptide of claim 1, wherein the recombinant polypeptide is a single-domain antibody.

4. The single-domain antibody of claim 3, wherein the single-domain antibody is a humanized single domain antibody.

5. The recombinant polypeptide of claim 1, wherein the recombinant polypeptide comprises a VHH sequence.

6. The recombinant polypeptide of claim 5, wherein the VHH sequence comprises camelized or llamaized framework regions of a human VH.

7. The recombinant polypeptide of claim 1, further comprising at least one therapeutic agent or imaging moiety.

8. The recombinant polypeptide of claim 7, wherein the recombinant polypeptide is either directly linked to the at least one therapeutic agent or imaging moiety, or is linked to the at least one therapeutic agent or imaging moiety via a linker or spacer.

9. The recombinant polypeptide of claim 8, wherein the linker comprises an amino acid sequence.

10. The recombinant polypeptide of claim 9, wherein the linker amino acid sequence comprises between about 1-25 amino acids, about 1-20 amino acids, about 1-15 amino acids, about 1-10 amino acids, about 1-5 amino acids, about 1-4 amino acids, about 1-3 amino acids, about 2 amino acids, or one (1) amino acid.

11. The recombinant polypeptide of claim 9, wherein the linker comprises an amino acid sequence selected from the group consisting of (GGS)n, (GGGS)n (SEQ ID NO:9), and (GGGGS)n (SEQ ID NO:10), wherein n=1-5.

12. The recombinant polypeptide of claim 7, wherein the at least one therapeutic agent comprises a therapeutic agent selected from the group consisting of chemotherapeutic agents, immunotherapeutic agents, radioactive agents, or a biologic agents.

13. The recombinant polypeptide of claim 7, wherein the at least one imaging moiety is selected from the group consisting of radiolabels, fluorescent labels, enzymatic labels, or PET imaging agents.

14. A pharmaceutical composition, comprising a therapeutically effective amount of the recombinant polypeptide of claim 1 and a pharmaceutically acceptable carrier.

15. A method of treating at least one condition mediated by HERV-K subtype HML-2, comprising administering the pharmaceutical composition of claim 14 to a patient in need of such treatment.

16. The method of claim 15, wherein the at least one condition mediated by HERV-K subtype HML-2 is selected from cancer (breast, brain, prostate, melanoma, germ cell tumors, ovarian, pancreatic, testes, glioblastoma, teratocarcinoma, medulloblastoma, lung, hepatocellular, colorectal, sarcoma, lymphoma, and/or metastases thereof), neurodegenerative diseases (ALS, Jacob Creutzfeldt Disease, Alzheimer's disease, and Frontotemporal dementia), or immune diseases (Rheumatoid arthritis, myalgic encephalomyelitis/chronic fatigue syndrome, and Lupus) that express HERV-K subtype HML-2.

17. An isolated nucleic acid molecule, comprising a nucleotide sequence encoding the recombinant polypeptide of claim 1.

18. An expression vector, comprising the nucleic acid molecule of claim 17.

19. An isolated host cell, comprising the nucleic acid molecule of claim 17.

20. An isolated host cell, comprising the expression vector of claim 18.

21. The isolated host cell of claim 19, wherein the host cell is a mammalian cell or an insect cell.

22. A method for making the recombinant polypeptide that specifically binds to one or more envelope epitopes of HERV-K subtype HML-2, comprising expressing in a host cell the isolated nucleic acid molecule of claim 17.

23. A method for making a recombinant polypeptide that specifically binds to one or more envelope epitopes of HERV-K subtype HML-2, comprising expressing in a host cell at least one nucleic acid molecule comprising a nucleotide sequence encoding a polypeptide that comprises:
(a) the amino acid sequence of SEQ ID NOs: 3 or 4, or a fragment thereof;
(b) an amino acid sequence that is at least 85% identical to the amino acid sequence of SEQ ID NOs: 3 or 4; or
(c) complementarity determining regions, CDR1, CDR2, and CDR3, wherein:
(i) CDR1 comprises the amino acid sequence of SEQ ID NO:5, or an amino acid sequence that is at least 85% identical to the amino acid sequence of SEQ ID NO:5;
(ii) CDR2 comprises the amino acid sequence of SEQ ID NO:6, or an amino acid sequence that is at least 85% identical to the amino acid sequence of SEQ ID NO:6; and
(iii) CDR3 comprises the amino acid sequence of SEQ ID NO:7, or an amino acid sequence that is at least 85% identical to the amino acid sequence of SEQ ID NO:7.

24. An in vitro method of detecting the presence of one or more envelope epitopes of HERV-K subtype HML-2, comprising the steps of:
(a) obtaining a sample from a subject;
(b) contacting the sample with the recombinant polypeptide of claim 1;
(c) detecting the binding of the recombinant polypeptide in the sample; and
(d) comparing the binding detected in step (c) with a standard,
wherein a difference in binding relative to the standard indicates the presence of one or more envelope epitopes of HERV-K subtype HML-2 in the sample.

25. A method of detecting the presence of one or more envelope epitopes of HERV-K subtype HML-2 in a patient, comprising the steps of:
(a) administering to the patient the recombinant polypeptide of claim 1;
(b) detecting the binding of the recombinant polypeptide in the patient; and
(c) comparing the binding detected in step (b) with a standard;
wherein a difference in binding relative to the standard indicates the presence of one or more envelope epitope of HERV-K subtype HML-2 in the patient.

26. A chimeric antigen receptor (CAR), wherein the CAR comprises the recombinant polypeptide of claim 1.

27. A chimeric antigen receptor (CAR), wherein the CAR comprises:
(a) an extracellular binding domain that specifically binds to one or more envelope epitopes of HERV-K subtype HML-2;
(b) a transmembrane domain; and
(c) at least one cytoplasmic signaling domain.

28-35. (canceled)

36. A population of T-cells, comprising the chimeric antigen receptor (CAR) of claim 27.

37. (canceled)

38. A pharmaceutical composition, comprising a population of T-cells expressing the CAR of claim 27, and a pharmaceutically acceptable carrier.

39-40. (canceled)

41. An isolated nucleic acid, comprising a nucleotide sequence encoding the CAR of claim 27.

42. A method of inducing a T-cell response in a subject suffering from at least one condition mediated by HERV-K subtype HML-2, wherein the method comprises administering to the subject a therapeutically effective amount of the population of T-cells of claim 36, wherein the administration induces an immune response to the at least one condition mediated by HERV-K subtype HML-2.

43. (canceled)


SEQUENCE LISTING (10 sequences)

SEQ ID NO: 1 - DNA, 528 bp, synthetic construct
  misc_feature 1..528: note = Synthetic
  source 1..528: mol_type = other DNA
atggcggaagtgcagctgcaggcttccgggggaggatttgtgcagccgggggggtcattgcgactgagctgcgccgcatccggatcaacatggtatctggatgcaatgggctggtttcgtcaggcccctggcaaggagagagagttcgtttccgccatctccgacctggacgacacagcacgttactacgctgacagcgtaaagggaagatttacaattagccgggataactccaaaaacacggtctatctccagatgaacagcctcagggccgaggacacagctacgtattactgtgcctatctgggtgcaacaatggcagagacatattggggacaggggacgcaggtaactgtgagtagcgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatag

SEQ ID NO: 2 - DNA, 471 bp, synthetic construct
  misc_feature 1..471: note = Synthetic
  source 1..471: mol_type = other DNA
atggctgaggtgcagttgcaagcctccggcggcggtttcgttcaacctggaggttctttgcgtttgtcttgcgccgcgtcagggtcgacttggtatttagatgcgatgggatggttccggcaagcaccagggaaagaacgcgaattcgtttcagcaattagcgatttagacgacacggcgcggtactacgctgattcagtaaagggccgcttcaccattagtcgtgataattcaaaaaatacagtatatcttcagatgaactctctgcgtgctgaggacaccgcaacgtactactgcgcttatcttggggccacaatggcagagacctattggggccagggaacacaagtcacggtcagcagtggagcggcccgcaaaaagagacggcagcggcggagaggagcagcggagcaaaaattaattagcgaagaggatcttaatggggctgcgcatcaccatcatcatcattag

SEQ ID NO: 3 - protein, 175 aa, synthetic construct
  REGION 1..175: note = Synthetic
MAEVQLQASGGGFVQPGGSLRLSCAASGSTWYLDAMGWFRQAPGKEREFVSAISDLDDTARYYADSVKGRFTISRDNSKNTVYLQMNSLRAEDTATYYCAYLGATMAETYWGQGTQVTVSSAAAHHHHHHGAAEQKLISEEDLNGAAEQKLISEEDLNGAAEQKLISEEDLNGAA

SEQ ID NO: 4 - protein, 156 aa, synthetic construct
  REGION 1..156: note = Synthetic
MAEVQLQASGGGFVQPGGSLRLSCAASGSTWYLDAMGWFRQAPGKEREFVSAISDLDDTARYYADSVKGRFTISRDNSKNTVYLQMNSLRAEDTATYYCAYLGATMAETYWGQGTQVTVSSGAARKKRRQRRRGAAEQKLISEEDLNGAAHHHHHH

SEQ ID NO: 5 - protein, 9 aa, synthetic construct
  REGION 1..9: note = Synthetic
STWYLDAMG

SEQ ID NO: 6 - protein, 15 aa, synthetic construct
  REGION 1..15: note = Synthetic
FVSAISDLDDTARYY

SEQ ID NO: 7 - protein, 10 aa, synthetic construct
  REGION 1..10: note = Synthetic
YLGATMAETY

SEQ ID NO: 8 - protein, 9 aa, synthetic construct
  REGION 1..9: note = Synthetic
RKKRRQRRR

SEQ ID NO: 9 - protein, 4 aa, synthetic construct
  REGION 1..4: note = Synthetic
GGGS

SEQ ID NO: 10 - protein, 5 aa, synthetic construct
  REGION 1..5: note = Synthetic
GGGGS